(19) United States
(12) Patent Application Publication
(10) Pub. No.: US 2024/0050441 A1
(43) Pub. Date: Feb. 15, 2024

(54) COMBINATION THERAPIES FOR THE TREATMENT OF CANCER

(71) Applicant: Erasca, Inc., San Diego, CA (US)

(72) Inventors: Leenus MARTIN, San Diego, CA (US); Leslie Harris BRAIL, Newton, MA (US); Robert Field SHOEMAKER, San Diego, CA (US)

(21) Appl. No.: 18/256,089

(22) PCT Filed: Dec. 10, 2021
(86) PCT No.: PCT/US2021/062912

§ 371 Date: Jun. 6, 2023

Related U.S. Application Data
(60) Provisional application No. 63/277,561, filed on Nov. 9, 2021.
(60) Provisional application No. 63/253,012, filed on Oct. 6, 2021.
(60) Provisional application No. 63/214,718, filed on Jun. 24, 2021.
(60) Provisional application No. 63/124,665, filed on Dec. 11, 2020.

(51) Int. Cl. A61K 31/5383; A61K 39/395; A61K 31/506; A61K 31/517; A61P 35/00

(52) U.S. Cl. CPC A61K 31/5383; A61K 39/3955; A61K 31/506; A61K 31/517; A61P 35/00

(57) ABSTRACT

The present disclosure provides methods of treating cancer with combination therapies of a SHP2 inhibitor and EGFR inhibitor.

### CROSS-REFERENCE

This application claims the benefit of U.S. Provisional Patent Application No. 63/124,665 filed Dec. 11, 2020; U.S. Provisional Patent Application No. 63/214,718 filed Jun. 24, 2021; U.S. Provisional Patent Application No. 63/253,012 filed Oct. 6, 2021; and U.S. Provisional Patent Application No. 63/277,561 filed Nov. 9, 2021; each of which is incorporated herein by reference in its entirety.

### BACKGROUND OF THE INVENTION

Src Homology-2 phosphatase (SHP2) is a non-receptor protein phosphatase ubiquitously expressed in various tissues and cell types (see reviews: Taj an M et al., Eur J Med Genet 2016 58(10):509-25; Grossmann KS et al., Adv Cancer Res 2010 106:53-89). SHP2 is composed of two Src homology 2 (N-SH2 and C-SH2) domains in its NH2 -terminus, a catalytic PTP (protein-tyrosine phosphatase) domain, and a C-terminal tail with regulatory properties. At the basal state, the intermolecular interactions between the SH2 domains and the PTP domain prevent the access of substrates to the catalytic pocket, keeping SHP2 into a closed, auto-inhibited conformation. In response to stimulation, SHP2 activating proteins bearing phosphor-tyrosine motifs bind to the SH2 domains, leading to exposure of active site and enzymatic activation of SHP2.

### SUMMARY OF THE INVENTION

The present embodiments disclosed herein generally relate to compositions and methods related to combination therapies to treat cancer utilizing a SHP2 inhibitor in conjunction with an EGFR inhibitor, including while providing an unexpected degree of synergy.

SHP2 plays important roles in fundamental cellular functions including proliferation, differentiation, cell cycle maintenance and motility. By dephosphorylating its associated signaling molecules, SHP2 regulates multiple intracellular signaling pathways in response to a wide range of growth factors, cytokines, and hormones. Cell signaling processes in which SHP2 participates include the RAS-MAPK (mitogen-activated protein kinase), the PI3K (phosphoinositol 3 -kinase)-AKT, and the JAK-STAT pathways.

SHP2 also plays a signal-enhancing role on this pathway, acting downstream of RTKs and upstream of RAS. One common mechanism of resistance for pharmacological inhibition of MAPK signaling involves activation of RTKs that fuel reactivation of the MAPK signaling. RTK activation recruits SHP2 via direct binding and through adaptor proteins. Those interactions result in the conversion of SHP2 from the closed (inactive) conformation to open (active) conformation. SHP2 is an important facilitator of RAS signaling reactivation that bypasses pharmacological inhibition in both primary and secondary resistance. Inhibition of SHP2 achieves the effect of globally attenuating upstream RTK signaling that often drives oncogenic signaling and adaptive tumor escape (see Prahallad, A. et al. Cell Reports 12, 1978-1985 (2015); Chen Y N, Nature 535, 148-152(2016)), which is incorporated herein by reference in its entirety for all of its teachings, including without limitation all methods, compounds, compositions, data and the like, for use with any of the embodiments and disclosure herein.

In addition to SHP2, epidermal growth factor receptor (EGFR), a transmembrane protein that is a receptor for members of the epidermal growth factor family of extracellular protein ligands, also operates upstream of the RAS pathway. The opportunity to target signal transduction pathways from multiple angles and potentially ameliorate feedback loops upstream of Ras via SHP2 and EGFR provides opportunities for developing methods that employ combination therapies. Described herein are various particular methods of and compositions related to the use of SHP2 and EGFR inhibitors.

In a first aspect, the present disclosure provides a method of treating a subject having cancer comprising administering to the subject a therapeutically effective amount of a compound of Formula I or its pharmaceutically acceptable salt:

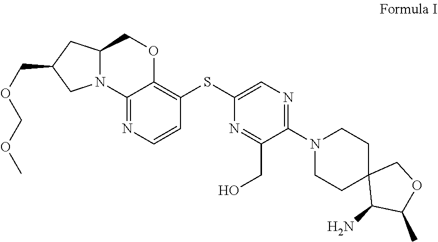 [SMILES: COCOC[C@H]1C[C@H]2COc3c(Sc4cnc(N5CCC6(CC5)CO[C@@H](C)[C@H]6N)c(CO)n4)ccnc3N2C1]

in combination with an EGFR inhibitor

In some embodiments, the EGFR in the subject is expressed constitutively.

In some embodiments, the cancer comprises an EGFR mutation selected from EGFR gene copy gain, EGFR gene amplification, chromosome 7 polysomy, EGFR L858R, EGFR exon 19 deletions/insertions (e.g., E746_A750del, E746_T751 delinsI, E746 T751delinsIP, E746_S752delinsA, E746_S752delinsV, E746_S752delinsV, L747_S752del, L747_T751del, and L747_P753delinsS), EGFR L861Q, EGFR G719C, EGFR G719S, EGFR G719A, EGFR V765A, EGFR T783A, EGFR exon 20 insertions (e.g., N771dup, N771_H773dup, and P772_H773dup), EGFR splice variants (e.g., Viii, Vvi, and Vii), EGFR A289D, EGFR A289T, EGFR A289V, EGFR G598A, EGFR G598V, EGFR T790M, and EGFR C797S.

In some embodiments, the cancer lung cancer.

In some embodiments, the cancer is an adenocarcinoma.

In some embodiments, the cancer is pancreatic ductal adenocarcinoma (PDAC).

In some embodiments, the EGFR inhibitor is selected from osimertinib, dacomitinib, lazertinib, nazartinib, neratinib, mobocertinib, afatinib, erlotinib, gefitinib, lapatinib, lifirafenib, amivantamab, cetuximab, panitumumab, necitumumab, mirzotamab clezutoclax, nimotuzumab and vandetanib.

In some embodiments, the EGFR inhibitor is osimertinib.

In some embodiments, the EGFR inhibitor is erlotinib.

In some embodiments, the EGFR inhibitor is gefitinib.

In some embodiments, the EGFR inhibitor is lapatinib.

In some embodiments, the EGFR inhibitor is neratinib.

In some embodiments, the EGFR inhibitor is afatnib.

In some embodiments, the method comprises administering a third MAPK pathway inhibitor.

In some embodiments, the administration is oral.

In some embodiments, the dosing of the compound of Formula I is in a range from 20 mg to 400 mg daily.

In some embodiments, the dosing of the EGFR inhibitor is in a range from 1 mg to 1500 mg daily.

In a second aspect, the present disclosure provides a method of treating lung cancer in a subject comprising orally administering to the subject a therapeutically effective amount of a compound of Formula I or its pharmaceutically acceptable salt:

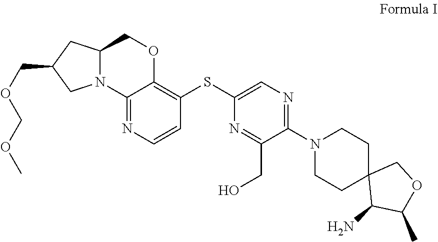 [SMILES: COCOC[C@H]1C[C@H]2COc3c(Sc4cnc(N5CCC6(CC5)CO[C@@H](C)[C@H]6N)c(CO)n4)ccnc3N2C1]

in combination with osimertinib.

In some embodiments, the compound of Formula I is administered once or twice daily.

In some embodiments, osimertinib is administered once or twice daily.

In some embodiments, the subject is a human.

In some embodiments, the lung cancer is non-small cell lung carcinoma.

In some embodiments, the lung cancer has an EGFR mutation.

In a third aspect, the present disclosure provides a kit comprising a compound of Formula I, or a pharmaceutically acceptable salt thereof, and an EGFR inhibitor.

In some embodiments, the compound of Formula I and the EGFR inhibitor are in separate packages.

In some embodiments, the kit further comprises instructions to administer the contents of the kit to a subject for the treatment of cancer.

In some embodiments, the EGFR inhibitor is one or more of osimertinib, dacomitinib, lazertinib, nazartinib, neratinib, mobocertinib, afatinib, erlotinib, gefitinib, lapatinib, lifirafenib, amivantamab, cetuximab, panitumumab, necitumumab, mirzotamab clezutoclax, nimotuzumab and vandetanib.

In a final aspect, the present disclosure provides a method of treating cancer in a subject comprising orally administering to the subject a therapeutically effective amount of a compound of Formula I or its pharmaceutically acceptable salt:

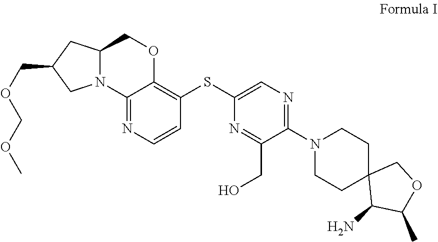 [SMILES: COCOC[C@H]1C[C@H]2COc3c(Sc4cnc(N5CCC6(CC5)CO[C@@H](C)[C@H]6N)c(CO)n4)ccnc3N2C1]

in combination with cetuximab.

In some embodiments, cetuximab is administered weekly.

In some embodiments, cetuximab is administered every other week.

In some embodiments, the cancer is squamous cell head and neck cancer (SCCHN).

In some embodiments, the cancer is pancreatic ductal adenocarcinoma (PDAC).

In some embodiments, the compound of Formula I is administered once or twice daily.

In some embodiments, the subject is a human.

In some embodiments, the compound or pharmaceutically acceptable salt thereof of Formula I is administered at a dose between about 20 mg and about 260 mg per day.

In some embodiments, the compound or pharmaceutically acceptable salt thereof of Formula I is administered at a dose between about 20 mg and about 60 mg per day.

In some embodiments, the compound or pharmaceutically acceptable salt thereof of Formula I is administered at a dose of about 40 mg per day or about 60 mg per day.

In some embodiments, the compound or pharmaceutically acceptable salt thereof of Formula I is administered at a dose between about 10 mg and about 100 mg twice per day.

In some embodiments, the compound or pharmaceutically acceptable salt thereof of Formula I is administered at a dose between about 20 mg and about 80 mg twice per day.

In some embodiments, the compound of Formula I, or a pharmaceutically acceptable salt thereof, is formulated as a pharmaceutical composition. In some embodiments, the compound of Formula I, or a pharmaceutically acceptable salt thereof, is formulated as an oral composition.

In some embodiments, the compound of Formula I, or a pharmaceutically acceptable salt thereof, is administered once or twice a day. In some embodiments, the compound of Formula I, or a pharmaceutically acceptable salt thereof, is administered once a day. In some embodiments, the compound of Formula I, or a pharmaceutically acceptable salt thereof, is administered twice a day. In some embodiments, the compound of Formula I, or a pharmaceutically acceptable salt thereof, is administered over a continuous 28-day cycle.

In some embodiments, the compound of Formula I, or a pharmaceutically acceptable salt thereof, is administered once a day in the amount of about 10 mg to about 140 mg.

In some embodiments, the compound of Formula I, or a pharmaceutically acceptable salt thereof, is administered once a day for a 3-week cycle, comprising 2 weeks of administration of the compound followed by 1 week of no administration of the compound.

In some embodiments, the compound of Formula I, or a pharmaceutically acceptable salt thereof, is administered once a day for a 4-week cycle, comprising 3 weeks of administration of the compound followed by 1 week of no administration of the compound.

In some embodiments, the compound of Formula I, or a pharmaceutically acceptable salt thereof, is administered over a period of 6 weeks. In some embodiments, the compound of Formula I, or a pharmaceutically acceptable salt thereof, is administered over a period of 8 weeks.

In some embodiments, the compound of Formula I, or a pharmaceutically acceptable salt thereof, is administered 3 times a week. In some embodiments, the compound of Formula I, or a pharmaceutically acceptable salt thereof, is administered on day 1, day 3, and day 5 of the week.

In some embodiments, the compound of Formula I, or a pharmaceutically acceptable salt thereof, is administered 4 times a week.

In some embodiments, the compound of Formula I, or a pharmaceutically acceptable salt thereof, is administered fora 3-week cycle, comprising 2 weeks of administration of the compound followed by 1 week of no administration of the compound.

In some embodiments, the compound of Formula I, or a pharmaceutically acceptable salt thereof, is administered for a 4-week cycle, comprising 3 weeks of administration of the compound followed by 1 week of no administration of the compound.

In some embodiments, the compound of Formula I, or a pharmaceutically acceptable salt thereof, is administered twice a day, two days per week. In some embodiments, the compound of Formula I, or a pharmaceutically acceptable salt thereof, is administered over a period of 8 weeks. In some embodiments, the compound of Formula I, or a pharmaceutically acceptable salt thereof, is administered on day 1 and day 2 of each week.

In some embodiments, the cancer is selected from lung cancer, stomach cancer, liver cancer, colon cancer, kidney cancer, breast cancer, pancreatic cancer, pancreatic ductal adenocarcinoma (PDAC) juvenile myelomonocytic leukemia, neurolastoma, melanoma, and acute myeloid leukemia.

### BRIEF DESCRIPTION OF THE DRAWINGS

FIG. 1A shows the data for the combination of the compound of Formula I and EGFR inhibitor osimertinib in cell line HCC827. This data indicates the combination of the compound of Formula I and EGFR inhibitor osimertinib exhibit synergy in vitro.

FIG. 1B shows the numerical data for the combination of the compound of Formula I and EGFR inhibitor osimertinib in cell line NCI-H820. This data indicates the combination of the compound of Formula I and EGFR inhibitor osimertinib exhibit synergy in vitro.

FIG. 2A shows a plot of percent activity versus inhibitor concentration (log M) in CAL-27 cells treated with the compound of Formula I alone (solid circles, Line 1), and in combination with 0.5 μg/ml (solid squares, Line 2), 1.0 μg/ml (solid circles, Line 3), or 2.5 μg/ml (solid squares, Line 4) of cetuximab.

FIG. 2B shows a bar chart of percent CTG activity that indicates cetuximab treatment alone decreased the cell viability by about 40%.

FIG. 3A shows a plot of the percent activity versus inhibitor concentration (log M) in SCC-9 cells treated with the compound of Formula I alone and in combination with 0.5 μg/ml (solid squares, Line 2), 1.0 μg/ml (solid circles, Line 3), or 2.5 μg/ml (solid squares, Line 4) of cetuximab.

FIG. 3B shows a bar chart of the percent CTG activity that indicates cetuximab treatment alone decreased the cell viability by about 15%.

FIG. 4A shows a plot of the percent activity versus inhibitor concentration (log M) in SCC-15 cells treated with the compound of Formula I alone and in combination with 1.0 μg/ml (solid squares, Line 2), 2.5 μg/ml (solid circles, Line 3), or 5.0 μg/ml (solid squares, Line 4) of cetuximab.

FIG. 4B shows a bar chart of the percent CTG activity that indicates cetuximab treatment alone decreased the cell viability by only <10%.

FIG. 5A shows a plot of the percent activity versus inhibitor concentration (log M) in SCC-25 cells treated with the compound of Formula I alone and in combination with 2.5 μg/ml (solid squares, Line 2), 5.0 μg/ml (solid circles, Line 3), or 10.0 μg/ml (solid squares, Line 4) of cetuximab.

FIG. 5B shows a bar chart of the percent CTG activity that indicates cetuximab treatment alone did not decrease the cell viability.

FIG. 6A shows an immunoblot of inhibition of ERK1/2 phosphorylation activity by approximately 50% with the compound of Formula I vs. DMSO.

FIG. 6B shows a bar chart of the quantified phosphorylated ERK1/2 bands, normalized by total ERK. The quantification results showed that treatment with the compound of Formula I alone or cetuximab alone decreased the ERK1/2 phosphorylation by about 50% relative to DMSO treated control cells, but the combination of the compound of Formula I and cetuximab showed about 80% inhibition of ERK1/2 phosphorylation in HPV-negative head and neck squamous cancer cell line, CAL 27.

FIG. 7A shows a table of Bliss synergy scores in HCC827/ER1 cell lines (erlotinib resistant) with a combination of the compound of Formula I and osimertinib.

FIG. 7B shows a table of Bliss synergy scores in HCC827 parental cell lines (erlotinib resistant) with a combination of the compound of Formula I and osimertinib.

FIG. 8 shows a graph of tumor volume over a period of treatment time with a regimen of the compound of Formula I alone, osimertinib alone, and the combination of the compound of Formula I and osimertinib in EGFR L858R/T790M mutant NSCLC PDX model LUN2355 -215.

FIG. 9 shows a graph of tumor volume over a period of treatment time with a regimen of the compound of Formula I alone, osimertinib alone, and the combination of the compound of Formula I and osimertinib in EGFR delE746_E749/T790M mutant and MET amplified NSCLC CDX model NCI-H820.

FIG. 10 shows a graph of tumor volume over a period of treatment time with a regimen of the compound of Formula I alone, osimertinib alone, and the combination of the compound of Formula I and osimertinib in EGFR L858R mutant and ERBB2 high expressing NSCLC PDX model LUN2005-143-9.

FIG. 11 shows a graph of tumor volume over a period of treatment time with a regimen of the compound of Formula I alone, osimertinib alone, and the combination of the compound of Formula I and osimertinib in EGFR L858R mutant NSCLC PDX model LUN2005-234.

FIG. 12 shows a graph of tumor volume over a period of treatment time with a regimen of the compound of Formula I alone, osimertinib alone, and the combination of the compound of

Formula I and osimertinib in EGFR L858R/T790M mutant NSCLC PDX model LUN2355 -128-33.

FIG. 13 shows a graph of tumor volume over a period of treatment time with a regimen of the compound of Formula I alone, osimertinib alone, and the combination of the compound of Formula I and osimertinib in EGFR^{ex19del} mutant erlotinib-resistant CDX model HCC827/ER1 (MET^{amp}) [E4957-U2101].

FIG. 14 shows a graph of tumor volume over a period of treatment time with a regimen of the compound of Formula I alone, cetuximab alone, and the combination of the compound of Formula I and cetuximab in RAS/RAF wild type PDX model CRC049.

FIG. 15 shows a graph of tumor volume over a period of treatment time with a regimen of the compound of Formula I alone, cetuximab alone, and the combination of the compound of Formula I and cetuximab in RAS/RAF wild type HPV-negative HNSCC CDX model FaDu.

### DETAILED DESCRIPTION OF THE INVENTION

### I. General

The present embodiments provide methods of treating a subject having cancer comprising administering to the subject a therapeutically effective amount of a compound of Formula I or its pharmaceutically acceptable salt:

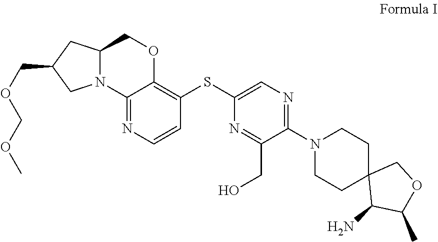 [SMILES: COCOC[C@H]1C[C@H]2COc3c(Sc4cnc(N5CCC6(CC5)CO[C@@H](C)[C@H]6N)c(CO)n4)ccnc3N2C1]

in combination with an EGFR inhibitor. The Examples below indicate a synergy for the combination that was unexpected. The combination therapies disclosed herein, employing the compound of Formula I or its pharmaceutically acceptable salt, can exhibit superior results compared to combinations of alternative SHP2 inhibitors used in combination with inhibitors of EGFR. Moreover, the combinations of the SHP2 inhibitor of Formula I and inhibitors of EGFR provide methods that allow the use of lower dosages of either agent used alone in a monotherapy, which can aid in reducing potential side effects. In particular, the combination therapies can be effective in cancer cells that express the have any EGFR mutation or overexpress EGFR. Accordingly, such treatments comport with the use of companion diagnostics to aid in proper patient population selection. These and other advantages will be recognized by those skilled in the art.

### II. Definitions

Unless specifically indicated otherwise, all technical and scientific terms used herein have the same meaning as commonly understood by those of ordinary skill in the art to which the embodiments are directed. In addition, any method or material similar or equivalent to a method or material described herein can be used in the practice of the embodiments herein. For purposes of the embodiments disclosed herein, the following terms are defined.

“A,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

“Pharmaceutically acceptable excipient” refers to a substance that aids the administration of an active agent to and absorption by a subject. Pharmaceutical excipients useful in the present embodiments include, but are not limited to, binders, fillers, disintegrants, lubricants, surfactants, coatings, sweeteners, flavors and colors. One of skill in the art will recognize that other pharmaceutical excipients are useful in the present embodiments.

“Treat”, “treating” and “treatment” refer to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject. In the context of the combination therapies disclosed herein, administration can be at separate times or simultaneous or substantially simultaneous.

“Co-administering” or “administering in combination with” as used herein refers to administering a composition described herein at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Coadministration is meant to include administration of the compounds on the same day, within the same week, and/or within the same treatment schedule. Compounds may have different administration schedules but still be co-administered if they are administered within the same treatment schedule. For example, palbociclib may be administered once a day for three weeks within a four week treatment schedule, and the compound of Formula I is co-administered with palbociclib if it is administered at any time within the four week treatment schedule.

“Therapeutically effective amount” refers to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins), each of which is incorporated herein by reference in its entirety for all of its teachings, including without limitation all methods, compounds, compositions, data and the like, for use with any of the embodiments and disclosure herein. In sensitized cells, the therapeutically effective dose can often be lower than the conventional therapeutically effective dose for non-sensitized cells.

“Inhibition,” “inhibits” and “inhibitor” refer to a compound that partially or completely blocks or prohibits or a method of partially or fully blocking or prohibiting, a specific action or function.

“Cancer” refers to all types of cancer, neoplasm or malignant tumors found in mammals (e.g. humans), including, without limitation, leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, pancreatic ductal adenocarcinoma (PDAC), or prostate cancer.

“EGFR inhibitor” refers to any inhibitor of wild-type EGFR or an EGFR mutant. EGFR mutations include, but are not limited to, any of those disclosed in U.S. Patent Publication No. 2018/0235968, which is incorporated herein by reference in its entirety. EGFR mutations include, without limitation, single nucleotide polymorphisms, exon insertion and deletions, polysomy, and the like. Specific examples of mutations include, without limitation, EGFR gene copy gain, EGFR gene amplification, chromosome 7 polysomy, EGFR L85 8R, EGFR exon 19 deletions/insertions (e.g., E746_A750del, E746_T751delinsI, E746_T751 delinsIP, E746_S752delinsA, E746_S752delinsV, E746_S752delinsV, L747_S752del, L747_T751del, and L747_P753delinsS), EGFR L861Q, EGFR L718Q, EGFR G719C, EGFR G719 S, EGFR G724S, EGFR G719A, EGFR V765A, EGFR T783A, EGFR exon 20 insertions (e.g., N771dup, N771_H773dup, and P772_H773dup), EGFR splice variants (e.g., Viii, Vvi, and Vii), EGFR A289D, EGFR A289T, EGFR A289V, EGFR G598A, EGFR G598V, EGFR S7681, EGFR T790M, EGFR C797 S, and EGFR C797S. In some embodiments, one or more of the mutations listed in this paragraph and elsewhere herein can be specifically excluded from the embodiments set forth herein, including without limitation, any methods, kits and compositions of matter, etc. Non-limiting examples of EGFR inhibitors include osimertinib, dacomitinib, lazertinib, nazartinib, neratinib, mob ocertinib, afatinib, erlotinib, gefitinib, lapatinib, lifirafenib, amivantamab, cetuximab, panitumumab, necitumumab, mirzotamab clezutoclax, nimotuzumab and vandetanib. Other EGFR inhibitors include those disclosed in U.S. Patent Publication Nos. 2020/0002279, 2019/0202920, and 2019/0167686 and International applications WO2012/061299, WO2019/067543, and WO2020/190765, each of which are incorporated herein by reference in their entirety. In some embodiments, one or more of the inhibitors listed in this paragraph and elsewhere herein, and those in the incorporated references, can be specifically excluded from one or more of the embodiments set forth herein, including without limitation, any methods, kits and compositions of matter, etc.

“Subject” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, horse, and other non-mammalian animals. In some embodiments, the patient is human.

### III. Dosing Methods

In some embodiments, the compound of Formula I, or a pharmaceutically acceptable salt thereof, is formulated as a pharmaceutical composition. In some embodiments, the compound of Formula I, or a pharmaceutically acceptable salt thereof, is formulated as an oral composition.

In some embodiments, the compound of Formula I, or a pharmaceutically acceptable salt thereof, is administered once or twice a day. In some embodiments, the compound of Formula I, or a pharmaceutically acceptable salt thereof, is administered once a day. In some embodiments, the compound of Formula I, or a pharmaceutically acceptable salt thereof, is administered twice a day. In some embodiments, the compound of Formula I, or a pharmaceutically acceptable salt thereof, is administered over a continuous 28-day cycle.

In some embodiments, the compound of Formula I, or a pharmaceutically acceptable salt thereof, is administered once a day in the amount of about 10 mg to about 140 mg.

In some embodiments, the compound of Formula I, or a pharmaceutically acceptable salt thereof, is administered once a day for a 3-week cycle, comprising 2 weeks of administration of the compound followed by 1 week of no administration of the compound.

In some embodiments, the compound of Formula I, or a pharmaceutically acceptable salt thereof, is administered once a day for a 4-week cycle, comprising 3 weeks of administration of the compound followed by 1 week of no administration of the compound.

In some embodiments, the compound of Formula I, or a pharmaceutically acceptable salt thereof, is administered over a period of 6 weeks. In some embodiments, the compound of Formula I, or a pharmaceutically acceptable salt thereof, is administered over a period of 8 weeks.

In some embodiments, the compound of Formula I, or a pharmaceutically acceptable salt thereof, is administered 3 times a week. In some embodiments, the compound of Formula I, or a pharmaceutically acceptable salt thereof, is administered on day 1, day 3, and day 5 of the week.

In some embodiments, the compound of Formula I, or a pharmaceutically acceptable salt thereof, is administered 4 times a week.

In some embodiments, the compound of Formula I, or a pharmaceutically acceptable salt thereof, is administered for a 3 -week cycle, comprising 2 weeks of administration of the compound followed by 1 week of no administration of the compound.

In some embodiments, the compound of Formula I, or a pharmaceutically acceptable salt thereof, is administered for a 4-week cycle, comprising 3 weeks of administration of the compound followed by 1 week of no administration of the compound.

In some embodiments, the compound of Formula I, or a pharmaceutically acceptable salt thereof, is administered twice a day, two days per week. In some embodiments, the compound of Formula I, or a pharmaceutically acceptable salt thereof, is administered over a period of 8 weeks. In some embodiments, the compound of Formula I, or a pharmaceutically acceptable salt thereof, is administered on day 1 and day 2 of each week.

In some embodiments, the cancer is selected from lung cancer, stomach cancer, liver cancer, colon cancer, kidney cancer, breast cancer, pancreatic cancer, juvenile myelomonocytic leukemia, neurolastoma, melanoma, and acute myeloid leukemia. In some embodiments, the cancer is pancreatic ductal adenocarcinoma (PDAC).

### IV. Combination Methods

In some embodiments, there are provided methods of treating a subject having cancer comprising administering to the subject a therapeutically effective amount of a compound of Formula I or its pharmaceutically acceptable salt:

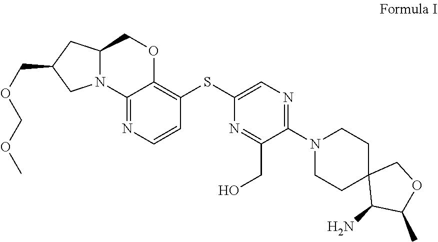 [SMILES: COCOC[C@H]1C[C@H]2COc3c(Sc4cnc(N5CCC6(CC5)CO[C@@H](C)[C@H]6N)c(CO)n4)ccnc3N2C1]

in combination with an EGFR inhibitor. As disclosed herein, a significant synergy was observed beyond that which had been anticipated for such a combination.

In some embodiments, the methods disclosed herein are suitable for the treatment of any cancer in which EGFR plays a role. In some embodiments, the cancer may be, for example, colorectal cancer (e.g., colon cancer, rectal cancer, etc.). In some embodiments, the cancer may be, for example, NSCLC (non-small cell lung cancer). In some embodiments, the cancer may be, for example, glioma, including glioblastoma. In some embodiments, the cancer is triple negative breast cancer. In some embodiments, the cancer is thyroid cancer. In some embodiments, the cancer may be, for example, head and neck squamous cell cancer. As will be appreciated by those skilled in the art, tumors may metastasize from a first or primary locus of tumor to one or more other body tissues or sites. In particular, metastases to the central nervous system (i.e., secondary CNS tumors), and particularly the brain (i.e., brain metastases), are well documented for tumors and cancers, such as breast, lung, melanoma, renal and colorectal. As such, the methods disclosed herein can be used for the treatment of metastases (i.e., metastatic tumor growth) to other organs as well.

In some embodiments, the method may include administering a third MAPK pathway inhibitor. Without being bound by theory, suppression of MAPK signaling in cancer cells can result in downregulation of PD-L1 expression and increase the likelihood that the cancer cells are detected by the immune system. Such third MAPK pathway inhibitors may be based on other mutations of proteins in the MAPK pathway. In some embodiments, any MAPK pathway inhibitor can be employed, including those targeting K-Ras, N-Ras, H-Ras, PDGFRA, PDGFRB, MET, FGFR, ALK, ROS1, TRKA, TRKB, TRKC, EGFR, IGFR1R, GRB2, SOS, ARAF, BRAF, RAF1, MEK1, MEK2, c-Myc, CDK4, CDK6, CDK2, ERK1, and ERK2. Exemplary MAPK pathway inhibitors include, without limitation, afatinib, osimertinib, erlotinib, gefitinib, lapatinib, neratinib, dacomitinib, vandetanib, cetuximab, panitumumab, nimotuzumab, necitumumab, trametinib, binimetinib, cobimetinib, selumetinib, ulixertinib, LTT462, and LY3214996. In some embodiments, one or more of the above-listed inhibitors can be specifically excluded from the embodiments set forth herein, including without limitation, any methods, kits and compositions of matter, etc.

The methods disclosed herein can be combined with other chemotherapeutic agents. Examples of such agents can be found in Cancer Principles and Practice of Oncology by V. T. Devita and S. Hellman (editors), 6th edition (Feb. 15, 2001), Lippincott Williams & Wilkins Publishers; which is incorporated herein by reference in its entirety for all of its teachings, including without limitation all methods, compounds, compositions, data and the like, for use with any of the embodiments and disclosure herein. A person of ordinary skill in the art would be able to discern which combinations of agents would be useful based on the particular characteristics of the drugs and the disease involved.

In some embodiments, the methods can include the co-administration of at least one cytotoxic agent. The term “cytotoxic agent” as used herein refers to a substance that inhibits or prevents a cellular function and/or causes cell death or destruction. Cytotoxic agents include, but are not limited to, radioactive isotopes (e.g., At211, I131, I125, Y90, Re186, Re188, Sm153, Bi212, P32, Pb212 and radioactive isotopes of Lu); chemotherapeutic agents; growth inhibitory agents; enzymes and fragments thereof such as nucleolytic enzymes; and toxins such as small molecule toxins or enzymatically active toxins of bacterial, fungal, plant or animal origin, including fragments and/or variants thereof.

Examples of cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A; inhibitors of fatty acid biosynthesis; cell cycle signaling inhibitors; HDAC inhibitors, proteasome inhibitors; and inhibitors of cancer metabolism.

Chemotherapeutic agents include chemical compounds useful in the treatment of cancer. Examples of chemotherapeutic agents include erlotinib (TARCEVA®, Genentech/OSI Pharm.), bortezomib (VELCADE®, Millennium Pharm.), disulfiram , epigallocatechin gallate , salinosporamide A, carfilzomib, 17-AAG(geldanamycin), radicicol, lactate dehydrogenase A (LDH-A), fulvestrant (FASLODEX®, AstraZeneca), sunitinib (SUTENT®, Pfizer/Sugen), letrozole (FEMARA®, Novartis), imatinib mesylate (GLEEVEC®., Novartis), finasunate (VATALANIB®, Novartis), oxaliplatin (ELOXATIN®, Sanofi), 5 -FU (5-fluorouracil), leucovorin, Rapamycin (Sirolimus, RAPAMUNE®, Wyeth), Lapatinib (TYKERB®, GSK572016, Glaxo Smith Kline), Lonafarnib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC 1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5 -alpha-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomy sins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6 azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumb erg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4 -hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3 -dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) rib ozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1 λ antibody genetically modified to recognize interleukin-12 p40 protein.

Chemotherapeutic agent also includes other “EGFR inhibitors,” which refers to compounds that bind to or otherwise interact directly with EGFR or its mutant forms and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn etal.) and variants thereof, such as chimerized 225 (C225 or Cetuximab; ERBUTIX□) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns etal., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA□ Genentech/OSI Pharmaceuticals); PD 183805 (CI 1033, 2 -propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine). Each of the above-described references is incorporated herein by reference in its entirety for all of its teachings, including without limitation all methods, compounds, compositions, data and the like, for use with any of the embodiments and disclosure herein.

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3 -d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca). Each of the above-described references is incorporated herein by reference in its entirety for all of its teachings, including without limitation all methods, compounds, compositions, data and the like, for use with any of the embodiments and disclosure herein.

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα)blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); miscellaneous investigational agents such as thioplatin, PS-341, phenylbutyrate, ET-18-OCH₃, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bc1-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with ox aliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

In certain embodiments, chemotherapeutic agents include, but are not limited to, doxorubicin, dexamethasone, vincristine, cyclophosphamide, fluorouracil, topotecan, interferons, platinum derivatives, taxanes (e.g., paclitaxel, docetaxel), vinca alkaloids (e.g., vinblastine), anthracyclines (e.g., doxorubicin), epipodophyllotoxins (e.g., etoposide), cisplatin, an mTOR inhibitor (e.g., a rapamycin), methotrexate, actinomycin D, dolastatin 10, colchicine, trimetrexate, metoprine, cyclosporine, daunorubicin, teniposide, amphotericin, alkylating agents (e.g., chlorambucil), 5-fluorouracil, campthothecin, cisplatin, metronidazole, and imatinib mesylate, among others. In other embodiments, a compound disclosed herein is administered in combination with a biologic agent, such as bevacizumab or panitumumab.

In certain embodiments, compounds disclosed herein, or a pharmaceutically acceptable composition thereof, are administered in combination with an antiproliferative or chemotherapeutic agent selected from any one or more of abarelix, aldesleukin, alemtuzumab, alitretinoin, allopurinol, altretamine, amifostine, anastrozole, arsenic trioxide, asparaginase, azacitidine, BCG live, bevacuzimab, fluorouracil, bexarotene, bleomycin, bortezomib, busulfan, calusterone, capecitabine, camptothecin, carboplatin, carmustine, cetuximab, chlorambucil, cladribine, clofarabine, cyclophosphamide, cytarabine, dactinomycin, darbepoetin alfa, daunorubicin, denileukin, dexrazoxane, docetaxel, doxorubicin (neutral), doxorubicin hydrochloride, dromostanolone propionate, epirubicin, epoetin alfa, elotinib, estramustine, etoposide phosphate, etoposide, exemestane, filgrastim, floxuridine, fludarabine, fulvestrant, gefitinib, gemcitabine, gemtuzumab, goserelin acetate, histrelin acetate, hydroxyurea, ibritumomab, idarubicin, ifosfamide, imatinib mesylate, interferon alfa-2a, interferon alfa-2b, irinotecan, lenalidomide, letrozole, leucovorin, leuprolide acetate, levamisole, lomustine, megestrol acetate, melphalan, mercaptopurine, 6-MP, mesna, methotrexate, methoxsalen, mitomycin C, mitotane, mitoxantrone, nandrolone, nelarabine, nofetumomab, oprelvekin, oxaliplatin, paclitaxel, palifermin, pamidronate, pegademase, pegaspargase, pegfilgrastim, pemetrexed disodium, pentostatin, pipobroman, plicamycin, porfimer sodium, procarbazine, quinacrine, rasburicase, rituximab, sargramostim, sorafenib, streptozocin, sunitinib maleate, talc, tamoxifen, temozolomide, teniposide, VM-26, testolactone, thioguanine, 6-TG, thiotepa, topotecan, toremifene, tositumomab, trastuzumab, tretinoin, ATRA, uracil mustard, valrubicin, vinblastine, vincristine, vinorelbine, zoledronate, or zoledronic acid.

In some embodiments, the dosing of the compound of Formula I can be in any suitable amount to treat the cancer. For example, the dosing could be a daily dosage of between 1 mg weight up to 500 mg. As an additional example, the daily dose could be in a range from about 20 mg to 400 mg (or any sub-range or sub-value there between, including endpoints). In some embodiments, the range of dosing of the compound of Formula I can be from 10 mg to 300 mg. In some embodiments, the range of dosing of the compound of Formula I can be from 10 mg to 100 mg. In some embodiments, the range of dosing of the compound of Formula I can be from 5 mg to 50 mg. The daily dosage can be achieved by administering a single administered dosage (e.g., QD) or via multiple administrations during a day (e.g., BID, TID, QID, etc.) to provide the total daily dosage. In some embodiments, the dosing of the EGFR inhibitor is any suitable amount. For example, it can be an amount in a range from 1 mg to 500 mg daily (or any sub-range or sub-value there between, including endpoints). Dosing of the EGFR inhibitor may be the same or less than the approved dosing for any given EGFR inhibitor and may depend on a given indication. For example, osimertinib for NSCLC may be administered from 20 to 100 mg daily, with commercial doses available in 40 mg and 80 mg. It will be appreciated that each of the recited ranges above can include any sub-range or sub-point therein, inclusive of endpoints. It will be appreciated that each of the recited ranges above can include any sub-range or sub-point therein, inclusive of endpoints. A common dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg. The amount of active ingredient that may be combined with the carrier materials to produce a single dosage form will vary depending upon the host treated and the particular mode of administration. In some embodiments, the administration is oral. For example, cetuximab for squamous cell head and neck cancer (SCCHN) may be administered from 500-100 mg/m² by intravenous infusion, with commercial doses available in 100 mg/50 ml and 200 mg/100 ml vials. It will be appreciated that each of the recited ranges above can include any sub-range or sub-point therein, inclusive of endpoints. It will be appreciated that each of the recited ranges above can include any sub-range or sub-point therein, inclusive of endpoints. A common dose range for adult humans is generally from 400 mg/m² for the initial infusion to 250 mg/m²/weekly infusion. Sub-ranges include 500 mg/m²/biweekly and 500 mg/m²/triweekly infusions. Sub-ranges include 250 mg/m²/weekly, 250 mg/m²/biweekly and 250 mg/m²/triweekly infusions. Sub-ranges include 200 mg/m²/weekly, 200 mg/m²/biweekly and 200 mg/m²/triweekly infusions. Sub-ranges include 150 mg/m²/weekly, 150 mg/m²/biweekly and 150 mg/m²/triweekly infusions. Sub-ranges include 100 mg/m²/weekly, 100 mg/m²/biweekly and 100 mg/m²/triweekly infusions. Sub-ranges include 500 mg/m²/120 minutes, 400 mg/m²/120 minutes, 250 mg/m²/60 minutes, 250 mg/m²/120 minutes, 200 mg/m²/60 minutes, 200 mg/m²/120 minutes, 150 mg/m²/60 minutes, 150 mg/m²/120 minutes, 100 mg/m²/60 minutes, or 100 mg/m²/120 minutes. In some embodiments, cetuximab may be administered for up to 54 months. In some embodiments, the administration is intravenous infusion.

In some embodiments, there are provided methods of treating colorectal cancer in a subject comprising orally administering to the subject a therapeutically effective amount of a compound of Formula I or its pharmaceutically acceptable salt in combination with an EGFR inhibitor, such as osimertinib. In some embodiments, the compound of Formula I is administered once or twice daily. In some embodiments, osimertinib is administered once or twice daily. The drugs can be co-administered as described herein, for example.

In some embodiments, there are provided methods of treating head and neck cancer (SCCHN) in a subject comprising orally administering to the subject a therapeutically effective amount of a compound of Formula I or its pharmaceutically acceptable salt in combination with an EGFR inhibitor, such as cetuximab. In some embodiments, the compound of Formula I is administered once or twice daily. In some embodiments, cetuximab is administered weekly. The drugs can be co-administered as described herein, for example.

In some embodiments, a patient having an EGFR mutation (a cancer having an EGFR mutation) is selected.

In some embodiments, a patient having NSCLC is selected. In some embodiments, a patient having NSCLC with an EGFR mutation is selected. In some embodiments, a patient having head and neck squamous cell carcinoma is selected. In some embodiments, a patient having head and neck squamous cell carcinoma with an EGFR mutation is selected. In some embodiments, the stages of head and neck squamous cell carcinoma is categorized in stages. In some embodiments, stage 0 generally refers to where the tumor is localized to the area it has started, no cancer cells are present in deeper layers of tissue, nearby structures, lymph nodes or distant sites. In some embodiments, stage 1 generally refers to where the primary tumor is 2 cm across or small and no cancer cells are present in deeper layers of tissue, nearby structures, lymph nodes or distant sites. In some embodiments, stage 2 generally refers to where the tumor measures 2-4 cm across and no cancer cells are present in deeper layers of tissue, nearby structures, lymph nodes or distant sites. In some embodiments, stage 3 generally refers to where the tumor is categorized by one of the following criteria (i) larger than 4 cm across and no cancer cells are present in deeper layers of tissue, nearby structures, lymph nodes or distant sites or (ii) the tumor is any size but has not grown into nearby structures or distant sites; cancer cells are present in one lymph node, which is located on the same side of the head or neck as the primary tumor and is smaller than 3 cm across. In some embodiments, the head and neck squamous cell carcinoma is level 2. In some embodiments, the head and neck squamous cell carcinoma is level 2/3. In some embodiments, the head and neck squamous cell carcinoma is level 3. In some embodiments, a patient having colorectal cancer is selected. In some embodiments, a patient having colorectal cancer with an EGFR mutation is selected.

In some embodiments, the cancer is human papillomavirus (HPV) negative. In some embodiments, the cancer does not have a KRAS mutation (wtKRAS). In some embodiments, the cancer does not have a NRAS mutation (wtNRAS). In some embodiments, the cancer does not have a BRAF mutation (wtBRAF). In some embodiments, the cancer is wtKRAS/wtNRAS/wtBRAF. In some embodiments, the cancer does not have a mutation in KRAS, NRAS or BRAF.

In some embodiments, the cancer has one or more acquired mutations. In some embodiments, the acquired mutation results from a first-line treatment. In some embodiments, the first-line treatment is an EGFR inhibitor. In some embodiments, the EGFR inhibitor is osimertinib. In some embodiments, the EGFR inhibitor is cetuximab. In some embodiments, the cancer is a solid tumor cancer. In some embodiments, the cancer is NSCLC.

In some embodiments, the acquired mutation is an acquired EGFR mutation. In some embodiments, the acquired EGFR mutation is C797X. In some embodiments, the acquired EGFR mutation is L718Q. In some embodiments, the acquired EGFR mutation is EGFR amplification. In some embodiments, the acquired EGFR mutation is G724S. In some embodiments, the acquired mutation is S768I.

In some embodiments, the acquired mutation is an acquired amplification mutation. In some embodiments, the acquired mutation is a MET gene amplification. In some embodiments, the acquired mutation is HER2 gene amplification.

In some embodiments, the acquired mutation is an acquired oncogenic fusion. In some embodiments, the acquired oncogenic fusion is SPTBN1 -ALK. In some embodiments, the acquired oncogenic fusion is RET fusion. In some embodiments, the acquired oncogenic fusion is BRAF fusion.

In some embodiments, the acquired mutation is an acquired MAPK-PI3K mutation. In some embodiments, the acquired MAPK-PI3K mutation is BRAF-V600E. In some embodiments, the acquired MAPK-PI3K mutation is PI3KCA. In some embodiments, the acquired MAPK-PI3K mutation is KRAS. In some embodiments, the acquired MAPK-PI3K mutation is HER2.

In some embodiments, the subject is a human. In some embodiments, the subject is a mammal other than a human, such as a primate, a rodent, a dog, a cat, or other small animal.

### Compositions

The compound of Formula I disclosed herein may exist as salts. The present embodiments include such salts, which can be pharmaceutically acceptable salts. Examples of applicable salt forms include hydrochlorides, hydrobromides, sulfates, methanesulfonates, nitrates, maleates, acetates, citrates, fumarates, tartrates (eg (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present embodiments contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present embodiments contain both basic and acidic functionalities that allow the compounds to be converted into either base or acid addition salts.

Other salts include acid or base salts of the compounds used in the methods of the present embodiments. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, and quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference in its entirety for all of its teachings, including without limitation all methods, compounds, compositions, data and the like, for use with any of the embodiments and disclosure herein.

Pharmaceutically acceptable salts include salts of the active compounds which are prepared with relatively nontoxic acids or bases, depending on the particular substituents found on the compounds described herein. When compounds of the present embodiments contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present embodiments contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19), which is incorporated herein by reference in its entirety for all of its teachings, including without limitation all methods, compounds, compositions, data and the like, for use with any of the embodiments and disclosure herein. Certain specific compounds of the present embodiments contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of the present embodiments can exist in unsolvated forms as well as solvated forms, including hydrated forms. In general, the solvated forms are equivalent to unsolvated forms and are encompassed within the scope of the present embodiments. Certain compounds of the present embodiments may exist in multiple crystalline or amorphous forms. In general, all physical forms are equivalent for the uses contemplated by the present embodiments and are intended to be within the scope of the present embodiments.

Certain compounds of the present embodiments possess asymmetric carbon atoms (optical centers) or double bonds; the enantiomers, racemates, diastereomers, tautomers, geometric isomers, stereoisomeric forms that may be defined, in terms of absolute stereochemistry, as (R)-or (S)- or, as (D)- or (L)- for amino acids, and individual isomers are encompassed within the scope of the present embodiments. The compounds of the present embodiments do not include those which are known in art to be too unstable to synthesize and/or isolate. The present embodiments is meant to include compounds in racemic and optically pure forms. Optically active (R)- and (S)-, or (D)- and (L)-isomers may be prepared using chiral synthons or chiral reagents, or resolved using conventional techniques.

Unless otherwise stated, the compounds of the present embodiments may also contain unnatural proportions of atomic isotopes at one or more of the atoms that constitute such compounds. For example, the compounds of the present embodiments may be labeled with radioactive or stable isotopes, such as for example deuterium (²H), tritium (³H), iodine-125 (¹²⁵I), fluorine-18 (¹⁸F), nitrogen-15 (¹⁵N), oxygen-17 (¹⁷O), oxygen-18 (¹⁸O), carbon-13 (¹³C), or carbon-14 (¹⁴C). All isotopic variations of the compounds of the present embodiments, whether radioactive or not, are encompassed within the scope of the present embodiments.

In addition to salt forms, the present embodiments provide compounds, which are in a prodrug form. Prodrugs of the compounds described herein are those compounds that readily undergo chemical changes under physiological conditions to provide the compounds of the present embodiments. Additionally, prodrugs can be converted to the compounds of the present embodiments by chemical or biochemical methods in an ex vivo environment. For example, prodrugs can be slowly converted to the compounds of the present embodiments when placed in a transdermal patch reservoir with a suitable enzyme or chemical reagent.

In some embodiments, there are provided pharmaceutical compositions comprising the compound of Formula I and a pharmaceutically acceptable excipient. In some embodiments, the pharmaceutical compositions are configured as an oral tablet preparation.

The compounds of the present embodiments can be prepared and administered in a wide variety of oral, parenteral and topical dosage forms. Oral preparations include tablets, pills, powder, dragees, capsules, liquids, lozenges, gels, syrups, slurries, suspensions, etc., suitable for ingestion by the patient. The compounds of the present embodiments can also be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present embodiments can be administered transdermally. The compounds of formula I disclosed herein can also be administered by in intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995), which is incorporated herein by reference in its entirety for all of its teachings, including without limitation all methods, compounds, compositions, data and the like, for use with any of the embodiments and disclosure herein. Accordingly, the present embodiments also provides pharmaceutical compositions including one or more pharmaceutically acceptable carriers and/or excipients and either a compound of formula I, or a pharmaceutically acceptable salt of a compound of formula I.

For preparing pharmaceutical compositions from the compounds of the present embodiments, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, surfactants, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”), which is incorporated herein by reference in its entirety for all of its teachings, including without limitation all methods, compounds, compositions, data and the like, for use with any of the embodiments and disclosure herein.

In powders, the carrier is a finely divided solid, which is in a mixture with the finely divided active component. In tablets, the active component is mixed with the carrier having the necessary binding properties and additional excipients as required in suitable proportions and compacted in the shape and size desired.

The powders, capsules and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other excipients, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients are carbohydrate or protein fillers including, but not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations disclosed herein can also be used orally using, for example, push-fit capsules made of gelatin, as well as soft, sealed capsules made of gelatin and a coating such as glycerol or sorbitol. Push-fit capsules can contain the compounds of formula I mixed with a filler or binders such as lactose or starches, lubricants such as talc or magnesium stearate, and, optionally, stabilizers. In soft capsules, the compounds of formula I may be dissolved or suspended in suitable liquids, such as fatty oils, liquid paraffin, or liquid polyethylene glycol with or without stabilizers.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of Formula I in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997, which is incorporated herein by reference in its entirety for all of its teachings, including without limitation all methods, compounds, compositions, data and the like, for use with any of the embodiments and disclosure herein. The pharmaceutical formulations disclosed herein can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The pharmaceutical formulations of the compound of Formula I disclosed herein can be provided as a salt and can be formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra; each of which is incorporated herein by reference in its entirety for all of its teachings, including without limitation all methods, compounds, compositions, data and the like, for use with any of the embodiments and disclosure herein.). The state of the art allows the clinician to determine the dosage regimen for each individual patient, GR and/or MR modulator and disease or condition treated.

Single or multiple administrations of the compound of Formula I formulations can be administered depending on the dosage and frequency as required and tolerated by the patient. The formulations should provide a sufficient quantity of active agent to effectively treat the disease state. Thus, in one embodiment, the pharmaceutical formulations for oral administration of the compound of Formula I is in a daily amount of between about 0.5 to about 30 mg per kilogram of body weight per day, including all sub-ranges and sub-values therein, inclusive of endpoints. In an alternative embodiment, dosages are from about 1 mg to about 20 mg per kg of body weight per patient per day are used. Lower dosages can be used, particularly when the drug is administered to an anatomically secluded site, such as the cerebral spinal fluid (CSF) space, in contrast to administration orally, into the blood stream, into a body cavity or into a lumen of an organ. Substantially higher dosages can be used in topical administration. Actual methods for preparing formulations including the compound of Formula I for parenteral administration are known or apparent to those skilled in the art and are described in more detail in such publications as Remington's, supra. See also Nieman, In “Receptor Mediated Antisteroid Action,” Agarwal, et al., eds., De Gruyter, New York (1987), which is incorporated herein by reference in its entirety for all of its teachings, including without limitation all methods, compounds, compositions, data and the like, for use with any of the embodiments and disclosure herein.

In some embodiments, co-administration includes administering one active agent within 0.5, 1, 2, 4, 6, 8, 10, 12, 16, 20, or 24 hours (or any sub -range of time or sub-value of time within a 24 hour period) of a second active agent. Co-administration includes administering two active agents simultaneously, approximately simultaneously (e.g., within about 1, 5, 10, 15, 20, or 30 minutes of each other (or any sub-range of time or sub-value of time from 0-30 minutes for example), or sequentially in any order. In some embodiments, co-administration can be accomplished by co-formulation, i.e., preparing a single pharmaceutical composition including both active agents. In some embodiments, the active agents can be formulated separately. In some embodiments, the active and/or adjunctive agents may be linked or conjugated to one another. At least one administered dose of drugs can be administered, for example, at the same time. At least one administered dose of the drugs can be administered, for example, within minutes or less than an hour of each other. At least one administered dose of drugs can be administered, for example, at different times, but on the same day, or on different days.

After a pharmaceutical composition including a compound of Formula I disclosed herein has been formulated in one or more acceptable carriers, it can be placed in an appropriate container and labeled for treatment of an indicated condition. For administration of the compounds of Formula I, such labeling would include, e.g., instructions concerning the amount, frequency and method of administration.

### Pharmaceutical Dosing

The dosage regimen for the compounds herein will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A clinical practitioner can determine and prescribe the effective amount of the drug required to prevent, counter, or arrest the progress of the disease or disorder.

By way of general guidance, the daily oral dosage of each active ingredient, when used for the indicated effects, will range between about 0.001 to about 1000 mg/kg of body weight, preferably between about 0.01 to about 100 mg/kg of body weight per day, and most preferably between about 0.1 to about 20 mg/kg/day. In some embodiments, a compound of Formula (I) may be administered at a dose of between about 10 mg/day and about 200 mg/day. In some embodiments, a compound of Formula (I) may be administered at a dose of about 10 mg/day, 20 mg/day, 30 mg/day, 40 mg/day, 50 mg/day, 60 mg/day, 70 mg/day, 80 mg/day, 90 mg/day, 100 mg/day, 110 mg/day, 120 mg/day, 130 mg/day, 140 mg/day, 150 mg/day, 160 mg/day, 170 mg/day, 180 mg/day, 190 mg/day, or 200 mg/day. The dose may be any value or subrange within the recited ranges.

Depending on the patient's condition and the intended therapeutic effect, the dosing frequency for the therapeutic agent may vary, for example, from once per day to six times per day. That is, the dosing frequency may be QD, i.e., once per day, BID, i.e., twice per day; TID, i.e., three times per day; QID, i.e., four times per day; five times per day, or six times per day. In another embodiment, dosing frequency may be BIW, i.e., twice weekly, TIW, i.e., three times a week, or QIW, i.e. four times a week.

Depending on the patient's condition and the intended therapeutic effect, the treatment cycle may have a period of time where no therapeutic agent is administered. As used herein, “interval administration” refers to administration of the therapeutic agent followed by void days or void weeks. For example, the treatment cycle may be 3 weeks long which includes 2 weeks of dosing of the therapeutic agent(s) followed by 1 week where no therapeutic agent is administered. In some embodiments, the treatment cycle is 4 weeks long which includes 3 weeks of dosing followed by 1 week where no therapeutic agent is administered.

The term “treatment cycle” as used herein, means a pre-determined period of time for administering the therapeutic agent. Typically, the patient is examined at the end of each treatment cycle to evaluate the effect of the therapy.

In one embodiment, each of the treatment cycle has about 3 or more days. In another embodiment, each of the treatment cycle has from about 3 days to about 60 days. In another embodiment, each of the treatment cycle has from about 5 days to about 50 days. In another embodiment, each of the treatment cycle has from about 7 days to about 28 days. In another embodiment, each of the treatment cycle has 28 days. In one embodiment, the treatment cycle has about 29 days. In another embodiment, the treatment cycle has about 30 days. In another embodiment, the treatment cycle has about 31 days. In another embodiment, the treatment cycle has about a month-long treatment cycle. In another embodiment, the treatment cycle is any length of time from 3 weeks to 8 weeks. In another embodiment, the treatment cycle is any length of time from 3 weeks to 6 weeks. In yet another embodiment, the treatment cycle is 3 weeks. In another embodiment, the treatment cycle is one month. In another embodiment, the treatment cycle is 4 weeks. In another embodiment, the treatment cycle is 5 weeks. In another embodiment, the treatment cycle is 6 weeks. In another embodiment, the treatment cycle is 7 weeks. In another embodiment, the treatment cycle is 8 weeks. The duration of the treatment cycle may include any value or subrange within the recited ranges, including endpoints.

As used herein, the term “co-administration” or “coadministration” refers to administration of (a) an additional therapeutic agent and (b) a compound of Formula (I), or a salt, solvate, ester and/or prodrug thereof, together in a coordinated fashion. For example, the co-administration can be simultaneous administration, sequential administration, overlapping administration, interval administration, continuous administration, or a combination thereof

In some embodiments, the dosing regimen for a compound of Formula (I) is once daily over a continuous 28-day cycle. In some embodiments, the once daily dosing regimen for a compound of Formula (I) may be, but is not limited to, 20 mg/day, 30 mg/day, 40 mg/day, 50 mg/day, 60 mg/day. Compounds of Formula (I) may be administered anywhere from 20 mg to 60 mg once a day. The dose may be any value or subrange within the recited ranges.

In some embodiments, the dosing regimen for a compound of Formula (I) is twice daily over a continuous 28-day cycle. In some embodiments, the twice daily dosing regimen for a compound of Formula (I) may be, but is not limited to, 10 mg/day, 20 mg/day, 30 mg/day, 40 mg/day, 50 mg/day, 60 mg/day, 70 mg/day, 80 mg/day, 90 mg/day, 100 mg/day. Compounds of Formula (I) may be administered anywhere from 20 mg to 80 mg twice a day. In some embodiments, compounds of Formula (I) may be administered anywhere from 10 mg/day to 100 mg/day. The dose may be any value or subrange within the recited ranges.

In some embodiments, the dosing regimen for a compound of Formula (I) may be once daily, anywhere from 20 mg to 60 mg per day for two weeks, followed by a one week break over a period of 6 weeks (e.g. 2 weeks on, 1 week off). In some embodiments, the dosing regimen for a compound of Formula (I) may be twice daily, anywhere from 10 mg to 100 mg twice a day for two weeks, followed by a one week break over a period of 6 weeks (e.g. 2 weeks on, 1 week off).

In some embodiments, the dosing regimen for a compound of Formula (I) may be once daily, anywhere from 20 mg to 60 mg per day for three weeks, followed by a one week break over a period of 8 weeks (e.g. 3 weeks on, 1 week off). In some embodiments, the dosing regimen for a compound of Formula (I) may be twice daily, anywhere from 10 mg to 100 mg twice a day for three weeks, followed by a one week break over a period of 8 weeks (e.g. 8 weeks on, 1 week off).

In some embodiments, the dosing regimen for a compound of Formula (I) may be twice daily on days 1 and 2, weekly for 8 weeks. In some embodiments, the dosing amount for compounds of Formula (I) may be, but is not limited to, 10 mg, 20 mg, 30 mg, 40 mg, 50 mg, 60 mg, 70 mg, 80 mg, 90 mg, 100 mg.

In some embodiments, the compound of Formula I, or a pharmaceutically acceptable salt thereof, is administered once a day fora 3-week cycle, comprising 2 weeks of administration of the compound followed by 1 week of no administration of the compound.

In some embodiments, the compound of Formula I, or a pharmaceutically acceptable salt thereof, is administered once a day fora 4-week cycle, comprising 3 weeks of administration of the compound followed by 1 week of no administration of the compound.

In some embodiments, the compound of Formula I, or a pharmaceutically acceptable salt thereof, is administered over a period of 6 weeks. In some embodiments, the compound of Formula I, or a pharmaceutically acceptable salt thereof, is administered over a period of 8 weeks.

In some embodiments, the compound of Formula I, or a pharmaceutically acceptable salt thereof, is administered 3 times a week. In some embodiments, the compound of Formula I, or a pharmaceutically acceptable salt thereof, is administered on day 1, day 3, and day 5 of the week.

In some embodiments, the compound of Formula I, or a pharmaceutically acceptable salt thereof, is administered 4 times a week.

In some embodiments, the compound of Formula I, or a pharmaceutically acceptable salt thereof, is administered for a 3-week cycle, comprising 2 weeks of administration of the compound followed by 1 week of no administration of the compound.

In some embodiments, the compound of Formula I, or a pharmaceutically acceptable salt thereof, is administered for a 4-week cycle, comprising 3 weeks of administration of the compound followed by 1 week of no administration of the compound.

In some embodiments, the compound of Formula I, or a pharmaceutically acceptable salt thereof, is administered twice a day, two days per week. In some embodiments, the compound of Formula I, or a pharmaceutically acceptable salt thereof, is administered over a period of 8 weeks. In some embodiments, the compound of Formula I, or a pharmaceutically acceptable salt thereof, is administered on day 1 and day 2 of each week.

When a compound of Formula I is administered multiple times a week, the dose may be administered on any day or combination of days within the week. For example, administration three times per week may include administration on days 1, 3, and 5; days 1, 2, and 3; 1, 3, and 5; and so on. Administration two days per week may include administration on days 1 and 2; days 1 and 3; days 1 and 4; days 1 and 5; days 1 and 6; days 1 and 7; and so on.

### Kits and Products

Some embodiments relate to kits and products that include the compound of Formula I and/or at least on EGFR inhibitor. For example, the kit or product can include a package or container with a compound of Formula I. Such kits and products can further include a product insert or label with approved drug administration and indication information, including how to use the compound of Formula I in combination with an EGFR inhibitor that is separately provided. The kits can be used in the methods of treating cancer as described herein.

In some aspects, the kits or products can include both a compound of Formula I and at least one EGFR inhibitor. In some embodiments, the EGFR inhibitor is osimertinib, for example. Such kits can include one or more containers or packages, which include one or both combination drugs together in a single container and/or package, or in separate packages/containers. In some instances, the two drugs are separately wrapped, but included in a single package, container or box. Such kits and products can further include a product insert or label with approved drug administration and indication information, including how to use the compound of Formula I in combination with an EGFR inhibitor. The kits can be used in the methods of treating cancer as described herein.

### EXAMPLES

### General Procedures

All starting materials and solvents were obtained either from commercial sources or prepared according to the literature citation.

### Example 1

Combination cellular proliferation assays: Cells (2000 cells per well) were plated onto 96-well plates in 100 μl cell culture medium. Cells were treated with the compound of Formula I and osimertinib at concentrations varying from 0 to 10 μM by using the Tecan D300e Digital Dispenser combination matrix protocol. At day 5, 50 μl of CellTiter-Glo (CTG) reagent (Promega) was added and the plates were incubated for 10 minutes with gentle shaking. After 10 minutes of incubation, the luminescent signal was determined according to the provider's instructions (Promega) and combination data was generated by the standard HSA model using Comb enefit software. The combination synergy was represented by positive numbers in results table. The negative numbers represent antagonism of the combination.

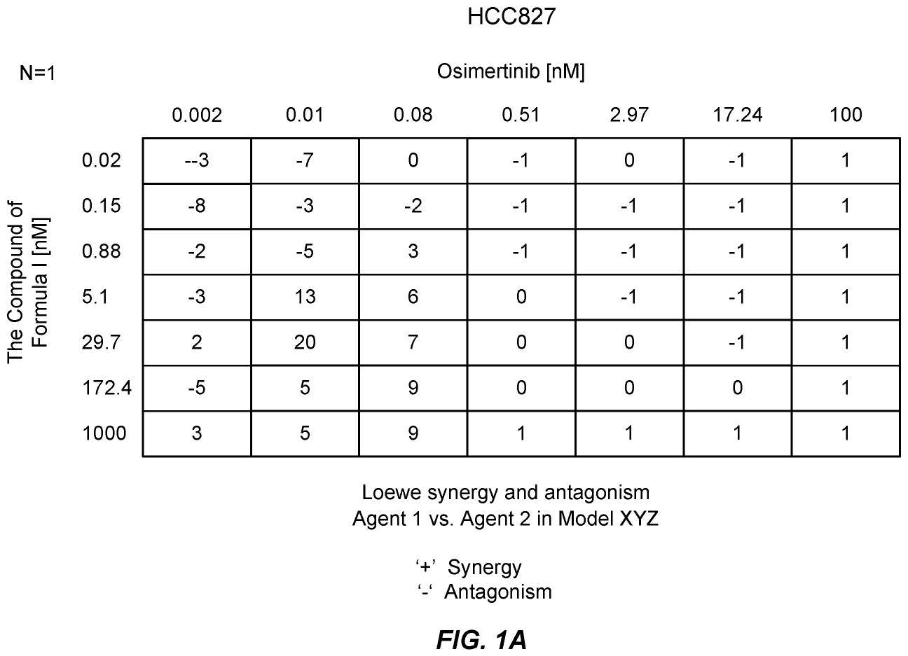

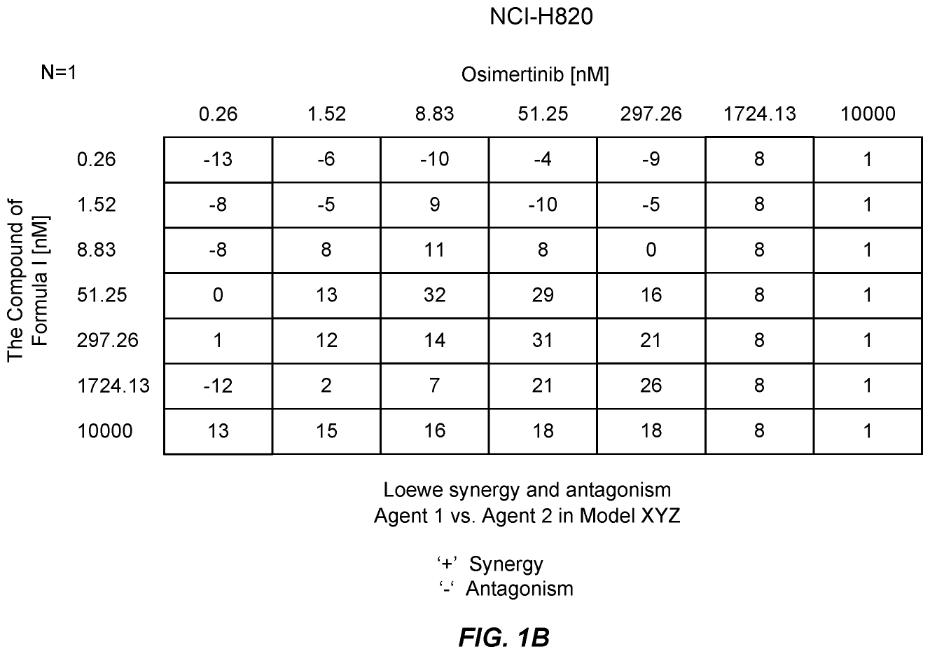

FIGS. 1A and 1B show data that indicate the compound of Formula I and EGFR inhibitor osimertinib in combination exhibit synergy in vitro, in EGFR mutant cell line HCC827, a lung adenocarcinoma having an acquired mutation in the EGFR tyrosine kinase domain (E746-A750 deletion), and NCI-H820, a lung adenocarcinoma cell line having an acquired mutation in the EGFR tyrosine kinase domain (T790M). FIG. 1A shows the numerical data for the compound of Formula I and EGFR inhibitor osimertinib in cell line HCC827. FIG. 1B shows the numerical data for the compound of Formula I and EGFR inhibitor osimertinib in cell line NCI-H820. The compound of Formula I and EGFRi (osimertinib) combination shows a strong synergistic viability effect.

### Example 2

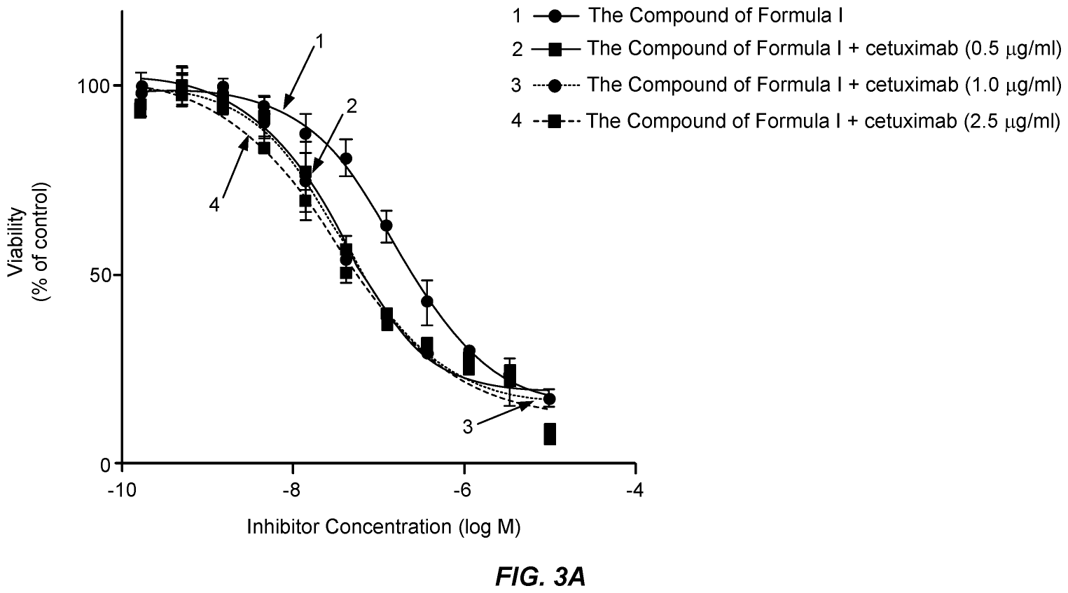

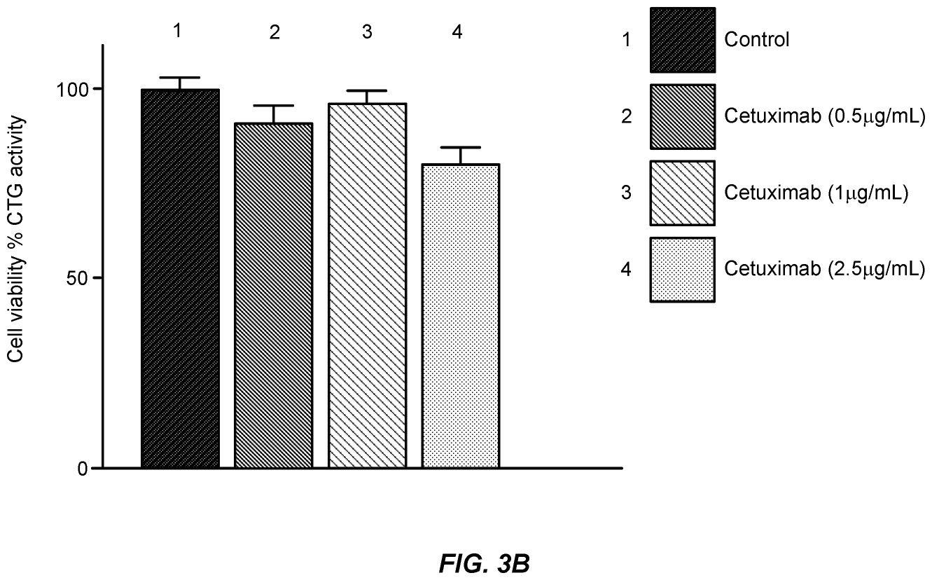

Results from these experiments are summarized in FIGS. 3 to 6 and described further below.

Table 1. Summary of cell lines and treatment with combination of the compound of Formula I and cetuximab.

TABLE 1

| | | The compound of Formula I + |
|---|---|---|
| No. | Cell line | cetuximab Combination |
| 1 | SCC-9 | Yes |
| 2 | SCC-15 | Yes |
| 3 | SCC-25 | Yes |
| 4 | SCC-4 | Yes |
| 5 | CAL 27 | Yes |

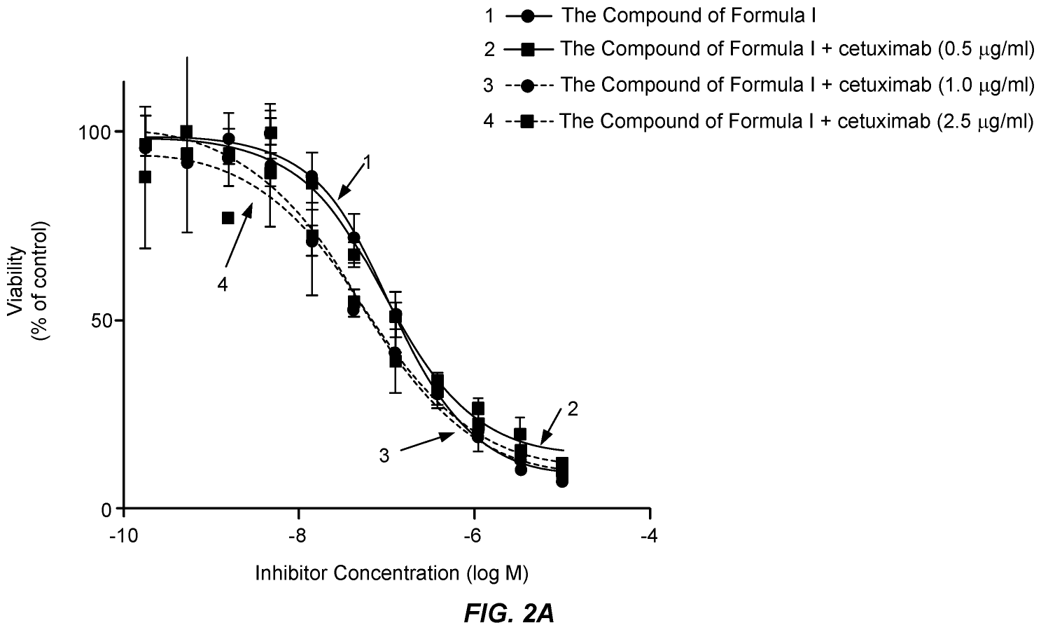

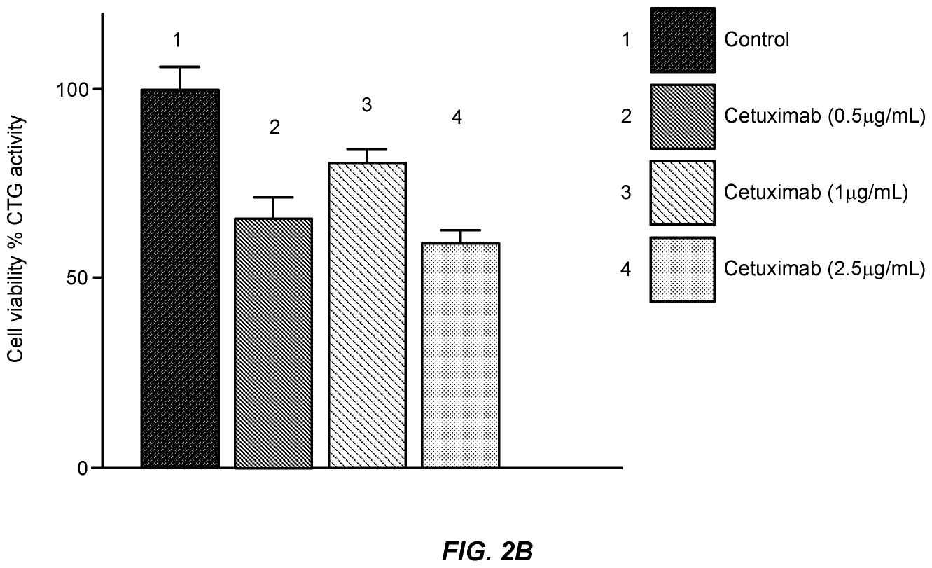

FIGS. 2A and 2B show treatment of CAL-27 cells with either the compound of Formula I alone, cetuximab alone, or the combination of the compound of Formula I and cetuximab. FIG. 2A shows a plot of percent activity versus inhibitor concentration (log M) in CAL-27 cells treated with the compound of Formula I alone (solid circles, Line 1) and in combination with 0.5 μg/ml (solid squares, Line 2), 1.0 μg/ml (solid circles, Line 3), or 2.5 μg/ml (solid squares, Line 4) of cetuximab.

FIG. 2B shows a bar chart of the percent CTG activity that indicates cetuximab treatment alone decreased the cell viability by about 40%.

Table 2. Co-treatment of cetuximab increased the compound of Formula I sensitivity in CAL-27 cells.

TABLE 2

| | | The compound of |
|---|---|---|
| No. | Treatment | Formula I IC50 (nM) |
| 1 | The compound of Formula I | 112 |
| 2 | The compound of Formula I + | 96 |
| | cetuximab (0.5 μg/ml) | |
| 3 | The compound of Formula I + | 49 |
| | cetuximab (1.0 μg/ml) | |
| 4 | The compound of Formula I + | 57 |
| | cetuximab (2.5 μg/ml) | |

FIGS. 3A and 3B show treatment of SCC-9 cells with either the compound of Formula I alone, cetuximab alone, or the combination of the compound of Formula I and cetuximab. FIG. 3A shows a plot of percent activity versus inhibitor concentration (log M) in SCC-9 cells treated with the compound of Formula I alone and in combination with 0.5 μg/ml (solid squares, Line 2), 1.0 μg/ml (solid circles, Line 3), or 2.5 μg/ml (solid squares, Line 4) of cetuximab. FIG. 3B shows a bar chart of percent CTG activity that indicates cetuximab treatment alone decreased the cell viability by about 15%.

Table 3. Co-treatment of cetuximab increased the compound of Formula I sensitivity in SCC-9 cells.

TABLE 3

| | | The compound of |
|---|---|---|
| No. | Treatment | Formula I IC50 (nM) |
| 1 | The compound of Formula I | 161 |
| 2 | The compound of Formula I + | 38 |
| | cetuximab (0.5 μg/ml) | |
| 3 | The compound of Formula I + | 35 |
| | cetuximab (1.0 μg/ml) | |
| 4 | The compound of Formula I + | 36 |
| | cetuximab (2.5 μg/ml) | |

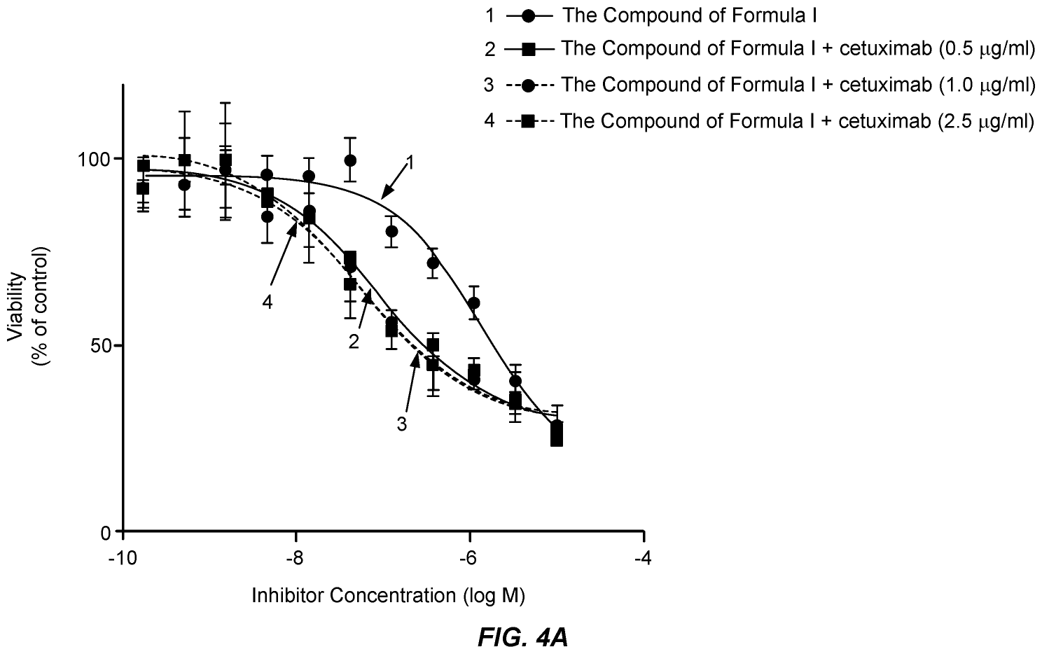

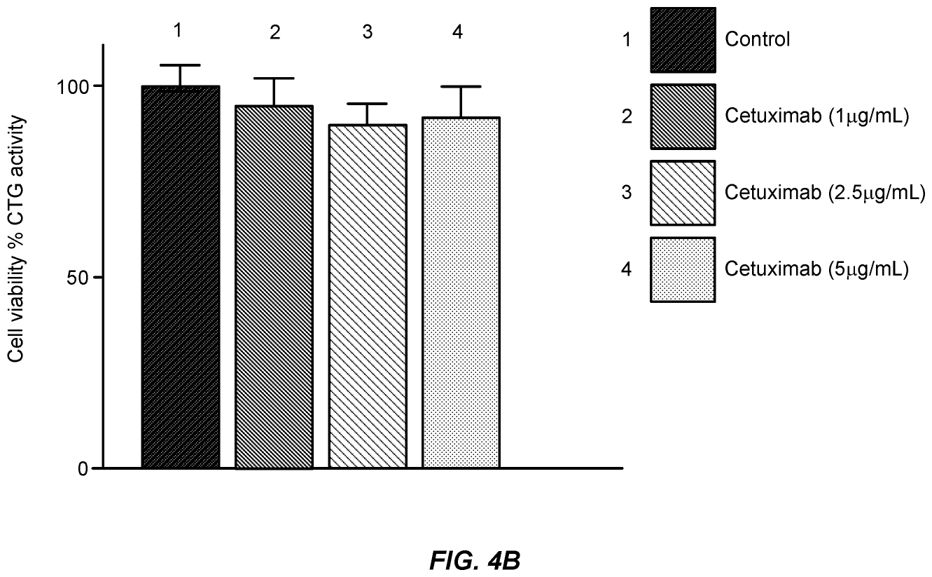

FIGS. 4A and 4B show treatment of SCC-15 cells with either the compound of Formula I alone, cetuximab alone, or the combination of the compound of Formula I and cetuximab. FIG. 4A shows a plot of percent activity versus inhibitor concentration (log M) in SCC-15 cells treated with the compound of Formula I alone and in combination with 1.0 μg/ml (solid squares, Line 2), 2.5 μg/ml (solid circles, Line 3), or 5.0 μg/ml (solid squares, Line 4) of cetuximab. FIG. 4B shows a bar chart of percent CTG activity that indicates cetuximab treatment alone decreased the cell viability by <10%.

Table 4. Co-treatment of cetuximab increased the compound of Formula I sensitivity in SCC-15 cells.

TABLE 4

| | | The compound of |
|---|---|---|
| No. | Treatment | Formula I IC50 (nM) |
| 1 | The compound of Formula I | 1406 |
| 2 | The compound of Formula I + | 91 |
| | cetuximab (0.5 μg/ml) | |
| 3 | The compound of Formula I + | 61 |
| | cetuximab (0.5 μg/ml) | |
| 4 | The compound of Formula I + | 55 |
| | cetuximab (0.5 μg/ml) | |

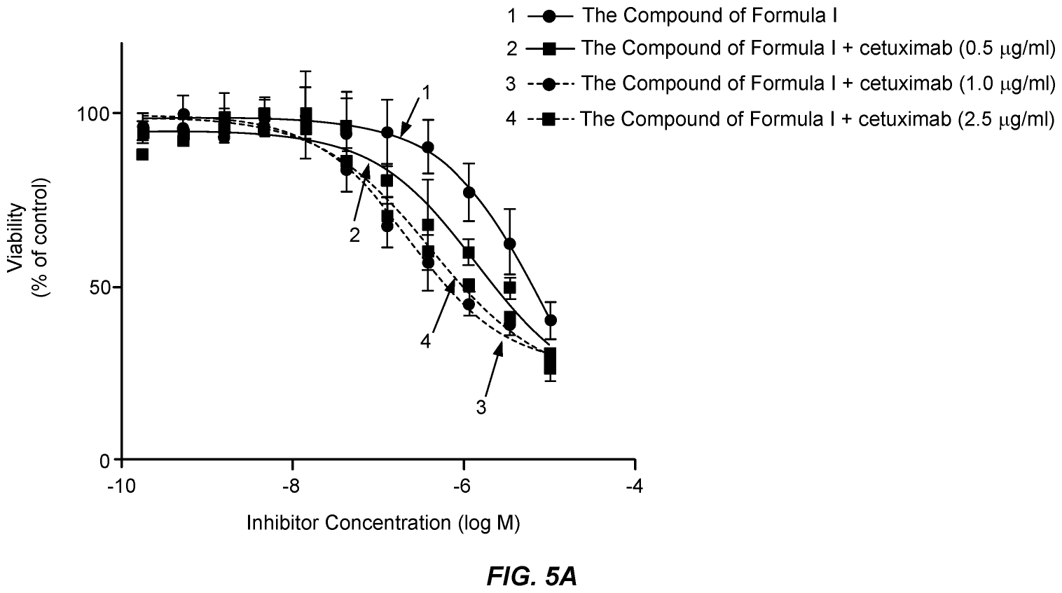

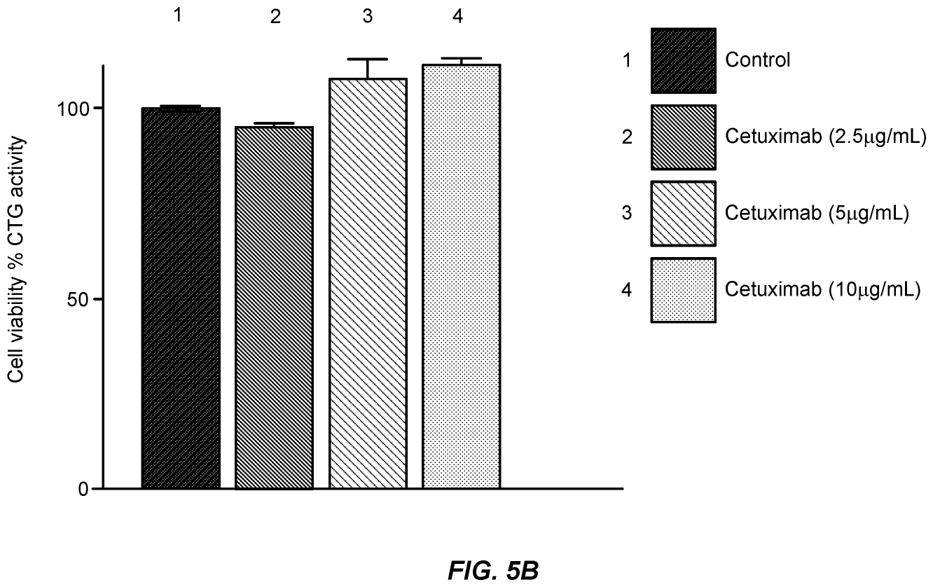

FIGS. 5A and 5B show treatment of SCC-25 cells with either the compound of Formula I alone, cetuximab alone, or the combination of the compound of Formula I and cetuximab. FIG. 5A shows a plot of percent activity versus inhibitor concentration (log M) in SCC-25 cells treated with the compound of Formula I alone and in combination with 2.5 μg/ml (solid squares, Line 2), 5.0 μg/ml (solid circles, Line 3), or 10.0 μg/ml (solid squares, Line 4) of cetuximab. FIG. 5B shows a bar chart of percent CTG activity that indicates cetuximab treatment alone did not decrease the cell viability.

Table 5. Co-treatment of cetuximab increased the compound of Formula I sensitivity in SCC-25 cells.

TABLE 5

| | | The compound of |
|---|---|---|
| No. | Treatment | Formula I IC50 (nM) |
| 1 | The compound of Formula I | 8046 |
| 2 | The compound of Formula I + | 1419 |
| | cetuximab (2.5 μg/ml) | |
| 3 | The compound of Formula I + | 225 |
| | cetuximab (5.0 μg/ml) | |
| 4 | The compound of Formula I + | 418 |
| | cetuximab (10.0 μg/ml) | |

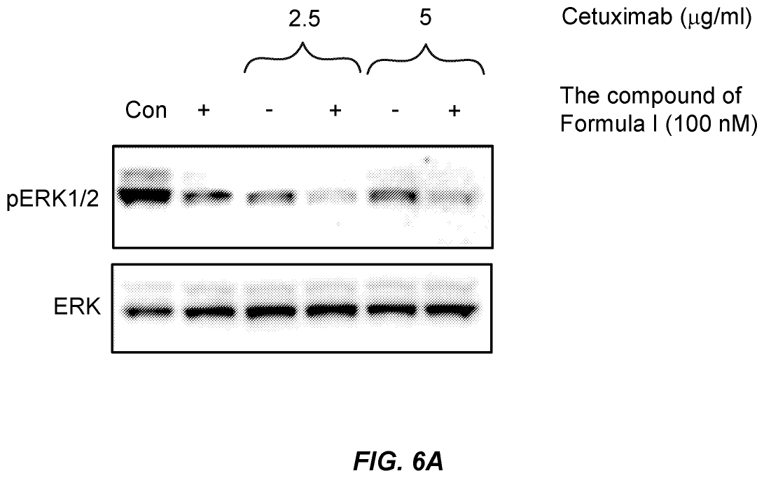

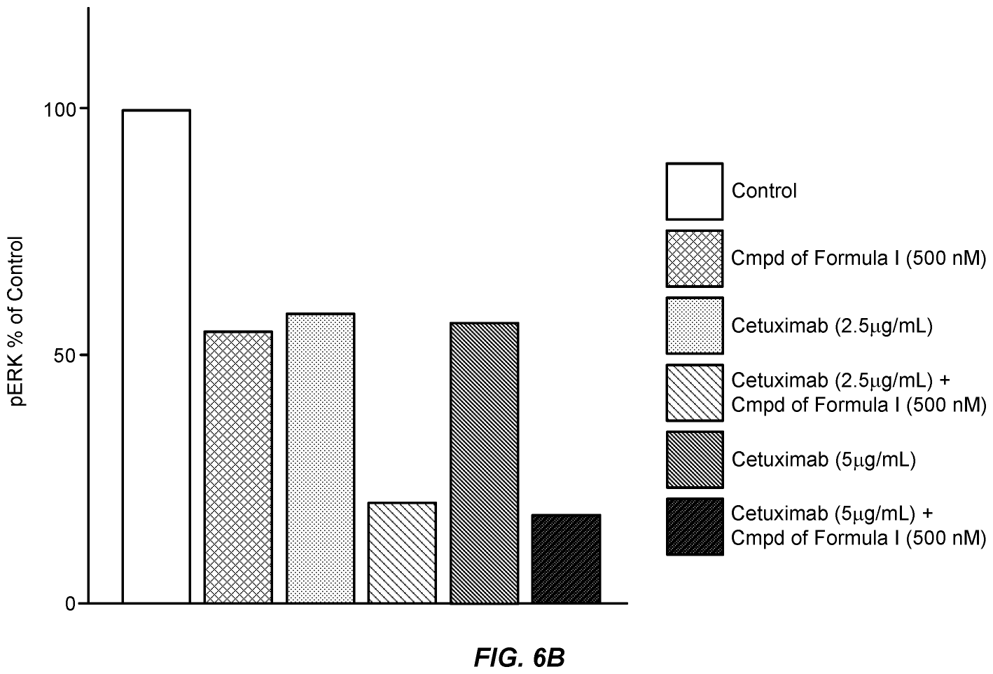

FIGS. 6A and 6B show inhibition of ERK1/2 phosphorylation in HPV negative CAL 27 cells with either the compound of Formula I alone, cetuximab alone, or the combination of the compound of Formula I and cetuximab. FIG. 6A shows an immunoblot of inhibition of ERK1/2 phosphorylation activity by approximately 50% by the compound of Formula I vs. DMSO. FIG. 6B shows the quantified phosphorylated ERK1/2 bands, normalized by total ERK. The quantification results showed that treatment of the compound of Formula I along or cetuximab alone decreased the ERK1/2 phosphorylation by about 50% relative to DMSO treated control cells, but the combination of the compound of Formula I and cetuximab showed about 80% inhibition of ERK1/2 phosphorylation in HPV-negative head and neck squamous cancer cell line, CAL 27.

### Example 3: Combination of the Compound of Formula I with Cetuximab Showed Combination Benefits on MAPK Signaling and Cell Viability in HPV-negative Head and Neck Squamous Cancer Cells

The purpose of this study is to determine the combination benefit on oncogenic MAPK signaling via SHP2 inhibition by the compound of Formula I and EGFR signaling via EGFR inhibition by cetuximab.

The cell lines used in the study were obtained from ATCC (CAL 27 #CRL-2095, SCC-15 #CRL-1623, SCC-25 #CRL-1628, SCC-4 #CRL-1624 and SCC-9 #CRL-1629). The cell line, CAL 27 was cultured in DMEM (Gib co #). SCC-4, SCC-15, SCC-25, SCC-9 cell lines was cultured in a 1:1 mixture of DMEM and Ham's F12 medium containing 1.2 g/L sodium bicarbonate, 2.5 mM L-glutamine, 15 mM HEPES and 0.5 mM sodium pyruvate supplemented with 400 ng/ml hydrocortisone, 90%; fetal bovine serum, 10% (Hyclone #SH-30071.03) and Penicillin/Streptomycin (Thermo Fisher #15070-063). The cells were maintained at 37° C./5% CO₂. Cells were washed in ice cold PBS (Thermo Fisher Scientific #10010-023) and lysed in RIPA Lysis and Extraction Buffer (Thermo Fisher Scientific #89900) supplemented with Halt Phosphatase and Protease Inhibitor (Thermo Fisher Scientific #78445). The membrane was blocked for non-specific proteins by Bovine Serum Albumin BSA (Cell Signaling Tech #9998). Antibodies for immunoblotting were as follows: phospho-p44/42 ERK1/2 (Cell Signaling Tech #4370), ERK2 antibody (Santa Cruz #sc-1647), anti-mouse IgG HRP-linked antibody (Cell Signaling Tech #7076), and anti-rabbit IgG HRP-linked antibody (Cell Signaling Tech #7074).

Cellular proliferation assay: Cells (2000 cells per well) were plated onto 96-well plates in 100 μl cell culture medium. Cells were treated with the compound of Formula I at concentrations varying from 0 to 10 μM and fixed cetuximab concentrations of 0.5, 1, 2.5, 5 and 10 μg/ml by using the Tecan D300e Digital Dispenser combination matrix protocol. At day 5, 50 μl of CellTiter-Glo (CTG) reagent (Promega) was added and the plates were incubated for 10 minutes with gentle shaking. After 10 minutes incubation, the luminescent signal was determined according to the provider's instruction (Promega) and combination data was generated by Comb enefit software.

Western blotting: Cells were lysed on ice for 10 minutes with Thermo Fisher RIPA lysis buffer with protease and phosphatase inhibitors. The cells were centrifuged at 4° for 10 minutes with a microcentrifuge. The supernatant was transferred to pre-chilled microcentrifuge tube and protein concentration of the lysate was measured using BCA method. Cell lysate supernatants of equal amount of proteins were used for immunoblotting.

The purpose of this study is to determine the combination benefit on oncogenic MAPK signaling via SHP2 inhibition by the compound of Formula I and EGFR signaling via EGFR inhibition by cetuximab. Three HPV-negative head and neck squamous cancer cell lines, CAL 27, SCC-4, SCC-9, SCC-15 and SCC-25 were split onto 96 well plates. After overnight incubation, the compound of Formula I and cetuximab were added to the cells by using the Tecan D300e Digital Dispenser combination matrix protocol. A CellTiter-Glo assay was executed after 5 days of incubation and the combination benefit was tested by increasing the compound of Formula I activity sensitivity by the addition of cetuximab. The combination data showed that cetuximab increased the sensitivity of the compound of Formula I in HPV-negative head and neck squamous cancer cell lines. (FIGS. 2-5).

The CAL 27 cells were treated with the compound of Formula I alone and also treated in combination with cetuximab. The compound of Formula I alone inhibited the cell proliferation of CAL 27 cells with an IC50 of 112 nM. The sensitivity of the compound of Formula I was increased when combined with cetuximab. The co-treatment of cetuximab sensitized the compound of Formula I activity dose dependently, and IC50 of the compound of Formula I decreased from 112 nM to 57 nM with co-treatment of 2.5 μg/ml of cetuximab (FIG. 2A and Table 2). Cetuximab alone treatment also decreased the cell viability by about 40% (FIG. 2B) and suggested that cetuximab treatment alone was effective in this cell line. The combination of the compound of Formula I and cetuximab showed more pronounced inhibition of cellular viability.

The SCC-9 cells were treated with the compound of Formula I alone and were also treated in combination with cetuximab. The compound of Formula I inhibited the cell proliferation of SCC-9 cells with an IC50 of 161 nM. The sensitivity of the compound of Formula I was increased when combined with cetuximab. The co-treatment of cetuximab sensitized the compound of Formula I activity dose dependently, and IC50 of the compound of Formula I decreased from 161 nM to 36 nM with co-treatment of 2.5 μg/ml of cetuximab (FIG. 3A and Table 3). However, there was no noticeable decrease in cell viability observed with up to 2.5 μg/ml of cetuximab alone treatment (FIG. 3B). The combination of the compound of Formula I and cetuximab showed more pronounced inhibition of cell viability.

The SCC-15 cells were treated with the compound of Formula I alone and were also treated in combination with cetuximab. The compound of Formula I inhibited the cell proliferation of SCC-15 cells with an IC50 of 1406 nM. The sensitivity of the compound of Formula I was increased when combined with cetuximab. The co-treatment of cetuximab sensitized the compound of Formula I activity dose dependently, and IC50 of the compound of Formula I decreased from 1406 nM to 55 nM with co-treatment of 5 μg/ml of cetuximab (FIG. 4A and Table 4). However, there was no noticeable decrease in cell viability observed with up to 5 μg/ml of cetuximab alone treatment (FIG. 4B). The combination of the compound of Formula I and cetuximab showed more pronounced inhibition of cellular viability.

The SCC-25 cells were treated with the compound Formula I alone and were also treated in combination with cetuximab. The compound of Formula I inhibited the cell proliferation of SCC-15 cells with an IC50 of 8046 nM. The sensitivity of the compound of Formula I was increased when combined with cetuximab. The co-treatment of cetuximab sensitized the compound of Formula I activity dose dependently, and IC50 of the compound of Formula I decreased from 1406 nM to 418 nM with co-treatment of 10 μg/ml of cetuximab (FIG. 5A and Table 5). However, there was no noticeable decrease in cell viability observed with up to 5 μg/ml of cetuximab alone treatment (FIG. 5B). The combination of the compound of Formula I and cetuximab showed more pronounced inhibition of cellular viability.

Combination of Formula I and cetuximab exhibited a robust inhibition of ERK1/2 phosphorylation in HPV-negative CAL 27 cells. The cell line CAL 27 was split onto a 6 well plate and treated separately with the compound of Formula I alone or cetuximab alone, and in combination as indicated (FIGS. 6A-6B). After 4 hours of treatment, cells were lysed and immunoblotted for phosphorylated ERK1/2. The immunoblot results showed that the compound of Formula I inhibited the ERK1/2 phosphorylation by about 50% relative to DMSO treated control cells. Cetuximab treatment exhibited an inhibition of ERK1/2 phosphorylation about 50%, and the inhibition was more pronounced (about 80%) when combined with the compound of Formula I (FIG. 6A). The phosphorylated ERK1/2 bands were quantified by using Bio-Rad Image Lab software and normalized by total ERK. The quantification results showed that treatment with the compound of Formula I alone or cetuximab alone decreased the ERK1/2 phosphorylation by about 50% relative to DMSO treated control cells, but the combination of the compound of Formula I and cetuximab showed about 80% inhibition of ERK1/2 phosphorylation in HPV-negative head and neck squamous cancer cell line, CAL 27 (FIG. 6B).

### Example 4: Open-Label Phase 1b/2 Study of Compound of Disclosure in Combination with Other Anti-Cancer Therapies in Patients with Advanced or Metastatic Solid Tumors

### Study Design

A compound of the disclosure (e.g., the compound of Formula I) in the form of a pharmaceutical composition is administered in combination with other cancer therapies in subjects having solid tumors that harbor specific molecular alterations in an open-label, multi-center clinical study. After the screening period, eligible subjects are enrolled and treated with the pharmaceutical composition comprising the compound of Formula I and another anti-cancer therapy until disease progression, unacceptable toxicity, or meeting another criterion for stopping treatment.

The study will evaluate the safety and tolerability of escalating doses of the compound of the disclosure (e.g., the compound of Formula I) when administered in combination with other cancer therapies; determine the maximum tolerated dose (MTD) and/or recommended dose (RD) of the compound of Formula I when administered in combination with other cancer therapies; characterize the pharmacokinetic (PK) profile of the compound when administered in combination with other cancer therapies; and to evaluate the antitumor activity when administered in combination with other cancer therapies.

### Outcome Measures

Primary Outcome Measures to be evaluated: (1) Dose Limiting Toxicities (DLT) (based on toxicities observed) (2) Maximum Tolerated Dose (MTD) (based on toxicities observed) (3) Recommended Dose (RD) (based on toxicities observed) (4) Adverse Events (AE) (incidence and severity of treatment-emergent AEs and serious AEs) (Time frame: assessed up to 24 months from time of first dose) (5) Plasma Concentration (Cmax) (Time Frame: Study Day up to Day 29) (6) Time to Achieve Cmax (Tmax) (Time Frame: Study Day up to Day 29) (7) Area Under the Curve (Area under the plasma concentration-time curve of compound of disclosure) (8) Half-life (Time Frame: Study Day 1 up to Day 29).

Secondary Outcome Measures to be Evaluated: (9) Objective response Rate (ORR) (based on assessment of radiographic imaging per RECIST version 1.1) (time frame: assessed up to 24 months from time of first dose) (10) Duration of Response (DOR) (based on assessment of radiographic imaging per RECIST version 1.1) (11) Time to Response (TTR) (based on assessment of radiographic imaging per RECIST version 1.1) (time frame: assessed up to 24 months from time of first dose).

Other Pre-specified Outcome Measures: (12) Pharmacodynamic Assessment (assessment of phosphorylated ERK (PERK) inhibition in PBMCs or tumor tissue by IHC or immunofluorescence (time framke: assessed up to 24 months from time of first dose.)

### Eligibility

Inclusion Criteria: (1) age ≥18 years (2) willing and able to give written informed consent (3) histologically or cytologically confirmed advanced or metastatic solid tumor (4) there is no available standard systemic therapy available for the patient's tumor histology and/or molecular biomarker profile; or standard therapy is intolerable, not effective, or not accessible (5) able to swallow oral medication (6) have Eastern Cooperative Oncology Group Performance Status (ECOG PS) of 0 or 1 (7) adequate cardiovascular, hematological, liver, and renal function and (7) willing to comply with all protocol-required visits, assessments, and procedures.

Exclusion Criteria: (1) previous treatment with a SHP2 inhibitor (2) documented PTPN11 mutations (3) receiving another study therapy or participated in a study of an investigational agent within 4 weeks of first dose (4) received prior palliative radiation within 7 days of cycle 1, day 1 (5) have primary central nervous system disease or known active CNS metastases and/or carcinomatous meningitis (6) prior surgery or gastrointestinal dysfunction that may affect drug absorption (7) active, clinically signification interstitial lung disease or pneumonitis (8) history of thromboembolic or cerebrovascular events within 12 weeks prior to first dose (9) history or current evidence of retinal vein occlusion (RVO or current risk factors for RVO (10) have any underlying medical condition, psychiatric condition, or social situation that, in the opinion of the Investigator, would comprise study administration as per protocol or compromise the assessment of Aes and (11) pregnant or breastfeeding or expecting to conceive or father children with the projected duration of the trial.

HPV Negative, Head and Neck Squamous Cell Carcinoma: Dose levels for once a day continuous dosing (QD) are 40 mg for the compound of Formula I, 20 to 60 mg QD, 40 mg QD or 60 mg QD. Dose levels for twice a day continuous dosing (BID) are 10-100 mg BID or 20 mg to 80 mg BID. Planned dosing schedule for QD or BID is 2 weeks on/1 week off. Dosing for Cetuximab is dosed every other week.

wtKRAS/wtNRAS/wtBRAF, Colorectal Cancer: Dose levels for once a day continuous dosing (QD) are 40 mg for the compound of Formula I, 20 to 60 mg QD, 40 mg QD or 60 mg QD. Dose levels for twice a day continuous dosing (BID) are 10-100 mg BID or 20 mg to 80 mg BID. Planned dosing schedule for QD or BID is 2 weeks on/1 week off. Dosing for Cetuximab is dosed every other week.

### Example 5—Combination Therapy of the Compound of Formula I and Osimertinib in HCC827 Cell Line and in Erlotinib Resistant Cell Line HCC827/ER1

### Materials and Methods

Human NSCLC cell line, HCC827 was purchased from ATCC. The EGFR exon 19 deletion mutant and MET amplified HCC827/ER1 cell line (Crown Bioscience UK) was derived from HCC827 (ATCC) at Crown Bioscience, by culturing the cells in the presence of escalating concentrations of erlotinib. HCC827/ER1 cells were cultured in medium containing RPMI-1640 plus 10% Fetal Bovine Serum (FBS) and 42 μM erlotinib at 37° C. in an atmosphere of 5% CO₂ in air. The medium was renewed routinely, and tumor cells were sub-cultured every 3 to 5 days at a confluence of 80% by trypsin-EDTA (Moores et al. 2016b).

### Cell Proliferation Assay:

The cells were harvested during the logarithmic growth period and were counted using Count-star. The cells were plated onto 96 well plates with a final cell density of 4×10³ cells/mL at a volume of 100 μL per well. After overnight incubation, the plated cells were equilibrated at RT for approximately 30 min. CellTiter-Glo reagent (50 μL) was added to each well and mixed for 5 min on an orbital shaker to induce cell lysis. The plates were incubated at RT for 20 min to stabilize luminescence signal. The luminescence signal for T0 was measured using an EnVision Multi Label Reader. For combination treatment, the drugs were prepared and dispensed at 1000× drug solution of each test article in each well simultaneously. The plates were incubated for 120 h in a humidified incubator at 37° C. with 5% CO₂ and then measured by a CTG assay. The plates were equilibrated at room temperature for approximately 30 min. The CellTiter-Glo (50 μL) was added to each well and plates were incubated for 5 min on an orbital shaker to induce cell lysis. The plates were then incubated at room temperature for 20 min to stabilize the luminescence signal (T5) and then measured using an EnVision Multi Label Reader.

### Synergy Calculation

The R package ‘SynergyFinder’ was used to calculate synergy score based on Loewe additivity model or Bliss independence model. The details of the calculation method is described below.

In a drug combination experiment where drug A at dose ×1 is combined with drug B at dose ×2, and the effect of this combination is measured as EABobs. In addition, the effect of drug A at dose ×1 is EA, while the effect of drug B at dose ×2 is EB.

The expected drug effect (EAB) is calculated based on either the Bliss model or the Loewe model:

Bliss model: EAB=EA+EB−EA*EB

Loewe model: A 4-parameter log-logistic model was used to fit the dose response curves for each drug, then a nonlinear equation was used to calculate EAB based on the parameters of log-logistic models.

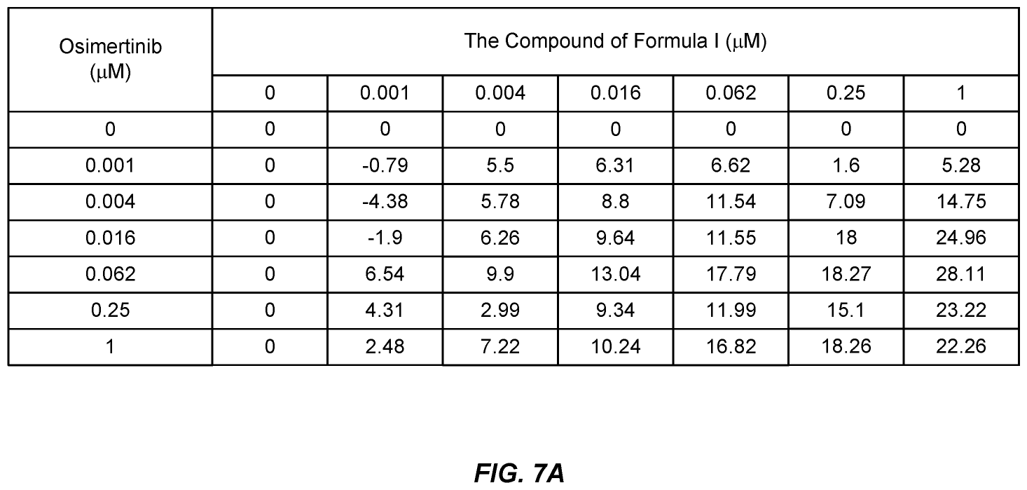

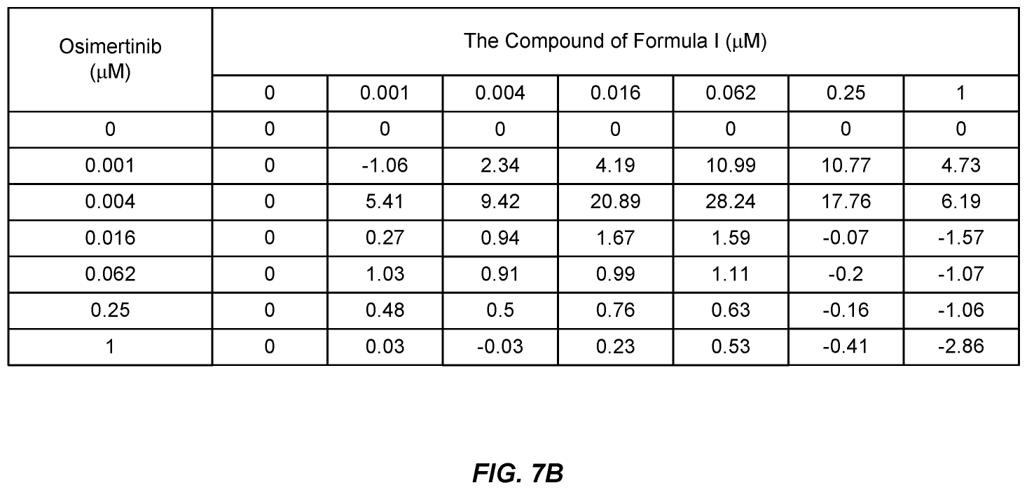

FIG. 7A shows a table of the Bliss synergy scores in HCC827/ER1 cell lines (erlotinib resistant) with a combination of the compound of Formula I and osimertinib.

FIG. 7B shows a table of the Bliss synergy scores in HCC827 cell lines (erlotinib resistant) with a combination of the compound of Formula I and osimertinib.

The percentage difference between EABob s and EAB is the synergy score for that combination.

### Example 6—Combination Therapy of the Compound of Formula I with Osimertinib in EGFR L858R/T790M Mutant NSCLC PDX Model LUN2355-215

### Materials

The vehicle/control article, 100 mM acetic acid in deionized water, with pH adjustment to 4.8-5.0, was prepared and stored under ambient conditions throughout the 18-day administration in mice.

The test article compound of Formula 1 was prepared in vehicle of 100 mM acetic buffer weekly and stored under ambient conditions. The combination agent osimertinib was prepared weekly in vehicle of 0.5% HPMC and 0.1% Tween 80 weekly and stored under ambient conditions.

Female Balb/c nude mice were purchased from the Beijing Vital River Laboratory Animal Technology Co., Ltd. Mice were hosted in special pathogen-free (SPF) environment of vivarium facility and acclimated to their new environment for at least 3 days prior to initiation of any experiments. Mice were between 6-8 weeks of age at the time of implantation.

All procedures related to animal handling, care, and treatment in this study were performed according to the protocols and guidelines approved by the Institutional Animal Care and Use Committee (IACUC) of GenenDesign. Animal facility and program was operated under the standard of Guide for the Care and Use of Laboratory Animals (NRC, 2011) and accredited by the Association for Assessment and Accreditation of Laboratory Animal Care (AAALAC). Specifically, all portions of this study performed at GenenDesign adhered to the study protocols reviewed and approved by IACUC and applicable standard operating procedures (SOPs).

### Preparation of PDXModel

The LUN2355-215 model was established for pre-clinical efficacy study at GenenDesign (Shanghai, China). This PDX model was derived from a 49-year-old male Chinese NSCLC patient. The EGFR mutations in the PDX model was confirmed by whole exome sequencing and PCR sequencing. Tumor fragments harvested from the PDX model were implanted subcutaneously in the right flanks of female Balb/c nude mice. Mice were anesthetized with isoflurane and anesthesia was maintained throughout the implantation procedure. Mouse skin was cleaned with appropriate surgical scrub and alcohol over the right flank. A small skin incision was made using the sharp end of the trochar and a 1.5 cm subcutaneous pocket along the right lateral chest wall was formed by blunt dissection with the stylet of a 10-12 g trochar needle. Tumor fragments (15-30 mm3) were placed into the trochar needle and advanced into the subcutaneous pocket in the right flank. Trochar incision was closed with suture or a wound clip that was removed one week after closure.

When tumor sizes reached 150-250 mm3 in volume, tumor-bearing mice were randomly divided into study groups with 8 mice in each group. The randomization date was denoted as treatment day 0.

### Treatment

Treatment started on the same day of randomization. The treatment start day was denoted as treatment day 0. Mice were dosed by oral administration of vehicle control solution, the compound of Formula I alone at 15 mg/kg QD, osimertinib alone at 15 mg/kg QD, and the combination of the compound of Formula I at 15 mg/kg QD and osimertinib at 15 mg/kg QD. The dosing volume was 5 mL/kg for each compound. Osimertinib was dosed one hour after the dosing of the compound of Formula I QD in the combination group. The study was terminated on treatment day 18 as defined in the study protocol.

### Results

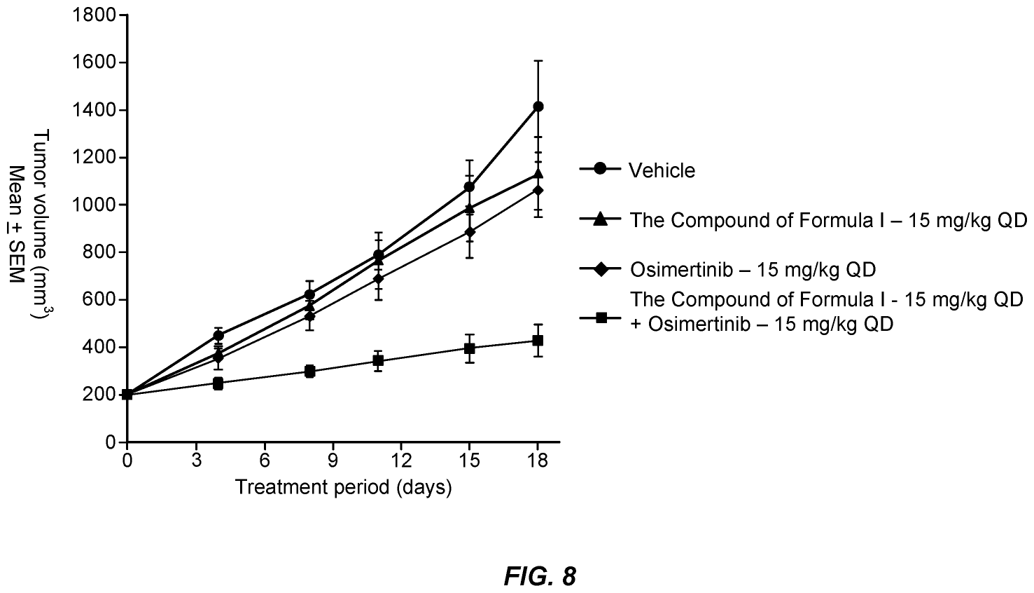

FIG. 8 shows a graph of tumor volume over a period of treatment time with a regimen of the compound of Formula I alone, osimertinib alone, and the combination of the compound of Formula I and osimertinib in EGFR L858R/T790M mutant NSCLC PDX model LUN2355-215. No significant body weight change was observed in the control and treatment groups.

### Conclusion

As shown in FIG. 8, the combination of the compound of Formula I and osimertinib demonstrated superior tumor growth inhibition relative to treatment with the compound of Formula I alone or treatment with osimertinib alone in EGFR L858R/T790M mutant NSCLC PDX model LUN2355-215.

### Example 7—Combination Therapy of the Compound of Formula I with Osimertinib in Osimertinib Resistant MET Amplified NSCLC CDX Model NCI-H820

### Materials

The vehicle/control article, 100 mM acetic acid in deionized water, with pH adjustment to 4.8-5.0, was prepared and stored under ambient conditions throughout the 28-day administration in mice.

The test article of the compound of Formula I was freshly prepared in vehicle of 100 mM acetic buffer weekly and stored under ambient conditions. The combination agent osimertinib was prepared weekly in vehicle of 0.5% HPMC and 0.1% Tween 80 weekly and stored under ambient conditions.

Female Balb/c nude mice were purchased from the Beijing Vital River Laboratory Animal Technology Co., Ltd. Mice were between 6-8 weeks of age at the time of implantation. Mice were hosted in a special pathogen-free (SPF) environment of vivarium facility and acclimated to their new environment for at least 3 days prior to initiation of any experiments.

All procedures related to animal handling, care, and treatment in this study were performed according to guidelines approved by the Institutional Animal Care and Use Committee (IACUC) of WuXi AppTec. During the study, the care and use of animals were conducted in accordance with the regulations of the Association for Assessment and Accreditation of Laboratory Animal Care (AAALAC). In addition, all portions of this study performed at WuXi AppTec adhered to the study protocols approved by the study director and applicable standard operating procedures (SOPs).

### Preparation of Xenograft Model

NCI-H820 is a human lung adenocarcinoma cell line that harbors an EGFR exon 19 deletion mutation, an EGFR T790M mutation, and a MET amplification. The NCI-H820 cell line was purchased from the American Type Culture Collection (ATCC® HTB-181™). NCI-H820 cells were cultured in medium containing RPMI-1640 plus 10% fetal bovine serum (FBS) and 1% antibiotic-antimycotic (AA), at 37° C. in an atmosphere of 5% CO2 in air. The medium was renewed every 2 to 3 days and tumor cells were routinely sub-cultured at a confluence of 80-90% by trypsin-EDTA. Cells growing in an exponential growth phase were harvested and counted for inoculation.

NCI-H820 tumor cells were implanted into mice subcutaneously. Briefly, 200 μL cell suspensions containing 10×10⁶ tumor cells mixed with 50% Matrigel were subcutaneously implanted into the right flank of the mouse using a syringe. Animal health and tumor growth were monitored daily. Tumor volume was measured twice a week by caliper when tumors were palpable and measurable. When tumor volumes reached a mean of 171 mm³ (range of 96-251 mm³), tumor-bearing mice were randomized into different groups with 8 mice in each group. The randomization date was denoted as treatment day 0.

### Treatment

Treatment started on the day after randomization. The treatment start day was denoted as treatment day 1. Mice were dosed by oral administration of vehicle control solution, the compound of Formula I alone at 30 mg/kg QD, and osimertinib alone at 15 mg/kg QD. One additional group received combination treatment of the compound of Formula I and osimertinib, dosing of the compound of Formula I at 15 mg/kg QD with osimertinib at 15 mg/kg QD. The dosing volume was 5 mL/kg for each compound. Osimertinib was dosed at one-hour after the dosing of the compound Formula I QD in combination groups. The study was terminated on treatment day 28 as being defined in the study protocol.

### Results

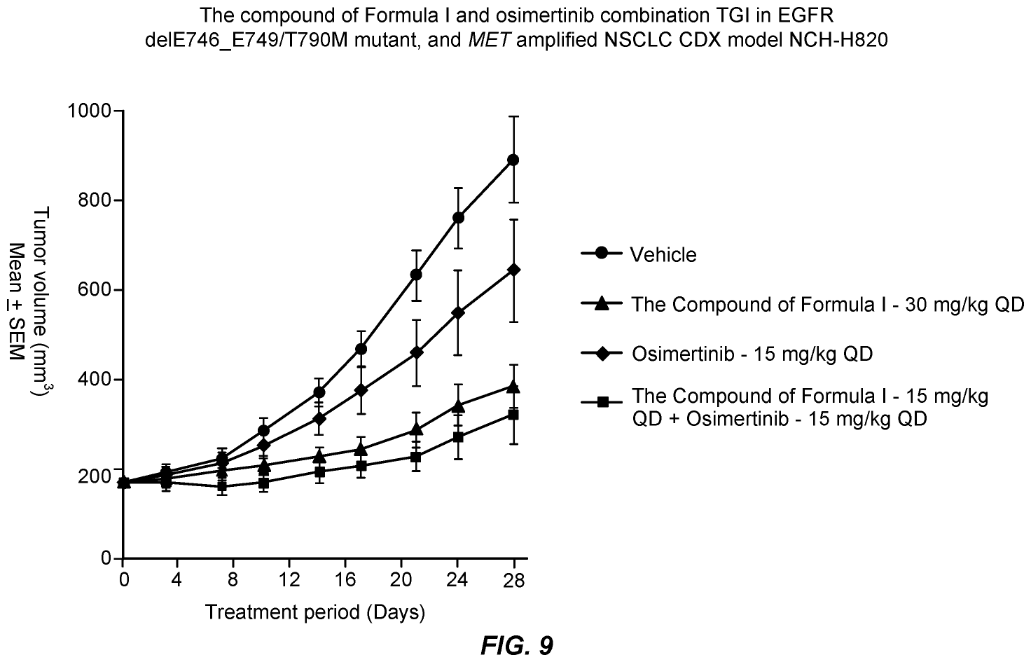

FIG. 9 shows a graph of tumor volume over a period of treatment time with a regimen of the compound of Formula I alone, osimertinib alone, and the combination of the compound of Formula I and osimertinib in EGFR delE746_E749/T790M mutant and MET amplified NSCLC CDX model NCI-H820. No significant body weight change was observed in the control and treatment groups.

### Conclusion

As shown in FIG. 9, the combination of the compound of Formula I and osimertinib demonstrated superior tumor growth inhibition relative to treatment with the compound of Formula I alone or treatment with osimertinib alone in EGFR delE746_E749/T790M mutant and MET amplified NSCLC CDX model NCI-H820.

### Example 8—Combination Therapy of the Compound of Formula I with Osimertinib in EGFR L858R Mutant and ERBB2 Overexpressing NSCLC PDX Model LUN2005-143-9

### Materials

The vehicle/control article, 100 mM acetic acid in deionized water, with pH adjustment to 4.8-5.0, was prepared and stored under ambient conditions throughout the 28-day administration in mice.

The test article of the compound of Formula I was prepared in vehicle of 100 mM acetic buffer weekly and stored under ambient conditions. The combination agent osimertinib was prepared weekly in vehicle of 0.5% HPMC and 0.1% Tween 80 weekly and stored under ambient conditions.

Female Balb/c nude mice were purchased from the Beijing Vital River Laboratory Animal Technology Co., Ltd. Mice were hosted in a special pathogen-free (SPF) environment of the vivarium facility and acclimated to their new environment for at least 3 days prior to initiation of any experiments. Mice were between 6-8 weeks of age at the time of implantation.

All procedures related to animal handling, care, and treatment in this study were performed according to the protocols and guidelines approved by the Institutional Animal Care and Use Committee (IACUC) of GenenDesign. The animal facility and program are operated under the standard of Guide for the Care and Use of Laboratory Animals (National Research Council, 2011) and accredited by the Association for Assessment and Accreditation of Laboratory Animal Care (AAALAC). Specifically, all portions of this study performed at GenenDesign adhered to the study protocols reviewed and were approved by IACUC and applicable standard operating procedures (SOPs).

Preparation of PDX model

The LUN2005-143-9 model was established for pre-clinical efficacy study at GenenDesign (Shanghai, China). The parental PDX model was derived from a 60-year-old male Chinese NSCLC patient with a tumor that harbored EGFRL858R mutation. The osimertinib resistant tumor, LUN2005-143-9 which has ERBB2 high expression, was derived from about 60 parental tumor-bearing mice after about 8 months of osimertinib treatment at 15 mg/kg QD. Tumor fragments harvested from the PDX model were implanted subcutaneously in the right flanks of female Balb/c nude mice. In order to do so, mice were anesthetized with isoflurane and anesthesia was maintained throughout the implantation procedure. Using aseptic surgical procedure, mouse skin was cleaned with an appropriate surgical scrub and alcohol over the right flank. A small skin incision was made using the sharp end of the trochar and a 1.5 cm subcutaneous pocket along the right lateral chest wall was formed by blunt dissection with the stylet of a 10-12 g trochar needle. Tumor fragments (15-30 mm³) were placed into the trochar needle and advanced into the subcutaneous pocket in the right flank. Trochar incision was closed with suture or a wound clip that was removed one week after closure. When tumor sizes reached 150-250 mm³ in volume, tumor-bearing mice were randomly divided into study groups with 8 mice in each group. The randomization date was denoted as treatment day 0.

### Treatment

Treatment started on the same day of randomization. The treatment start day was denoted as treatment day 0. Mice were dosed by oral administration of vehicle control solution, osimertinib alone at 15 mg/kg QD, and the compound of Formula I alone at 15 mg/kg QD. One additional group received the combination treatment of the compound of Formula I with osimertinib, with dosing of the compound of Formula I at 15 mg/kg QD and osimertinib at 15 mg/kg QD. The dosing volume was 5 mL/kg for each compound. Osimertinib was dosed one hour after the dosing of the compound Formula I QD dose in the combination group. The study was terminated on treatment day 28 as defined in the study protocol.

### Results

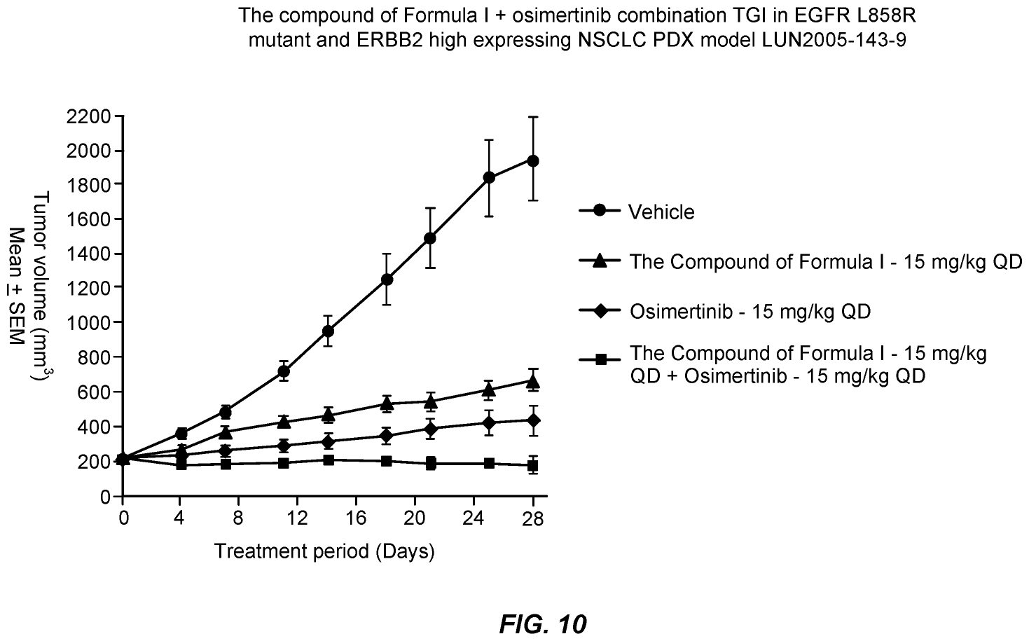

FIG. 10 shows a graph of tumor volume over a period of treatment time with a regimen of the compound of Formula I alone, osimertinib alone, and the combination of the compound of Formula I and osimertinib in EGFR L85 8R Mutant and ERBB2 Overexpressing NSCLC PDX Model LUN2005-143-9. No significant body weight change was observed in the control and treatment groups.

### Conclusion

As shown in FIG. 10, the combination of the compound of Formula I and osimertinib demonstrated superior tumor growth inhibition relative to treatment with the compound of Formula I alone or treatment with osimertinib alone in EGFR L858R Mutant and ERBB2 Overexpressing NSCLC PDX Model LUN2005-143-9.

### Example 9—Combination Therapy of the Compound of Formula I with Osimertinib in EGFR L858R mutant NSCLC PDX model LUN2005-234

### Materials

The vehicle/control article, 100 mM acetic acid in deionized water, with pH adjustment to 4.8-5.0, was prepared and stored under ambient conditions throughout the 24 -day administration in mice.

The test article of the compound of Formula I was prepared in vehicle of 100 mM acetic buffer weekly and stored under ambient conditions. The combination agent osimertinib was prepared weekly in vehicle of 0.5% HPMC and 0.1% Tween 80 weekly and stored under ambient conditions.

Female Balb/c nude mice were purchased from the Beijing Vital River Laboratory Animal Technology Co., Ltd. Mice were hosted in a special pathogen-free (SPF) environment of the vivarium facility and acclimated to their new environment for at least 3 days prior to initiation of any experiments. Mice were between 6-8 weeks of age at the time of implantation.

All procedures related to animal handling, care, and treatment in this study were performed according to the protocols and guidelines approved by the Institutional Animal Care and Use Committee (IACUC) of GenenDesign. The animal facility and program are operated under the standard of Guide for the Care and Use of Laboratory Animals (National Research Council, 2011) and accredited by the Association for Assessment and Accreditation of Laboratory Animal Care (AAALAC). Specifically, all portions of this study performed at GenenDesign adhered to the study protocols reviewed and were approved by IACUC and applicable standard operating procedures (SOPs).

Preparation of PDX model

The LUN2005 -234 model was established for pre-clinical efficacy study at GenenDesign (Shanghai, China). The parental PDX model was derived from a 60-year-old male Chinese NSCLC patient with a tumor that harbored EGFRL858R mutation. The osimertinib resistant tumor, LUN2005-234 was derived from about 60 parental tumor-bearing mice after about 7 months of osimertinib treatment at 15 mg/kg QD. Tumor fragments harvested from the PDX model were implanted subcutaneously in the right flanks of female Balb/c nude mice. To do so, mice were anesthetized with isoflurane and anesthesia was maintained throughout the implantation procedure. Using aseptic surgical procedure, mouse skin was cleaned with an appropriate surgical scrub and alcohol over the right flank. A small skin incision was made using the sharp end of the trocar and a 1.5 cm subcutaneous pocket along the right lateral chest wall was formed by blunt dissection with the stylet of a 10-12 g trocar needle. Tumor fragments (15-30 mm³) were placed into the trocar needle and advanced into the subcutaneous pocket in the right flank. Trocar incision was closed with suture or a wound clip which was removed one week after closure. When mean tumor volume reached 205 mm³ (tumor sizes ranged between 150-250 mm 3) tumor-bearing mice were randomly divided into study groups with 8 mice in each group. The randomization date was denoted as treatment day 0.

### Treatment

Treatment started on the same day of randomization. The treatment start day was denoted as treatment day 0. Mice were dosed by oral administration of vehicle control solution, osimertinib alone at 15 mg/kg QD, and the compound of Formula I alone at 15 mg/kg QD. One additional group received the combination treatment of the compound of Formula I with osimertinib, with dosing of the compound of Formula 1 at 15 mg/kg QD and dosing of osimertinib at 15 mg/kg QD. The dosing volume was 5 mL/kg for each compound. Osimertinib was dosed one hour after the dosing of the compound of Formula I QD dose in the combination group. The study was terminated on treatment day 24 as defined in the study protocol.

### Results

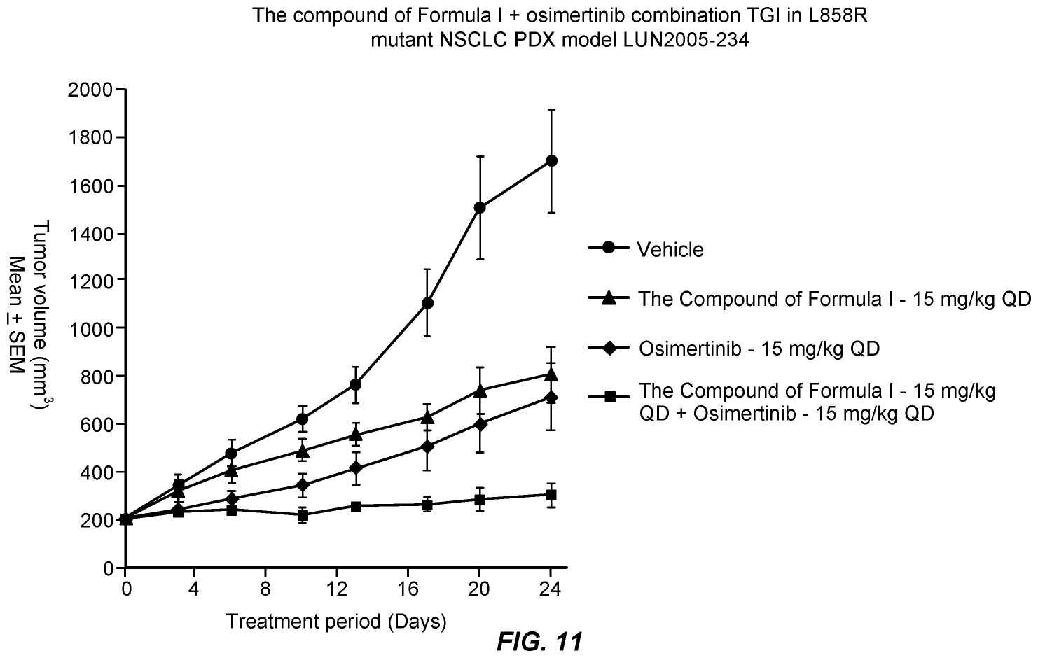

FIG. 11 shows a graph of tumor volume over a period of treatment time with a regimen of the compound of Formula I alone, osimertinib alone, and the combination of the compound of Formula I and osimertinib in EGFR L858R mutant NSCLC PDX model LUN2005-234. No significant body weight change was observed in the control and treatment groups.

### Conclusion

As shown in FIG. 11, the combination of the compound of Formula I and osimertinib demonstrated superior tumor growth inhibition relative to treatment with the compound of Formula I alone or treatment with osimertinib alone in EGFRL858R mutant NSCLC PDX model LUN2005-234.

### Example 10—Combination Therapy of the Compound of Formula I with Osimertinib in EGFR L858R/T790M mutant NSCLC PDX model LUN2355-128-33

### Materials

The vehicle/control article, 100 mM acetic acid in deionized water, with pH adjustment to 4.8-5.0, was prepared and stored under ambient conditions throughout the 20-day administration in mice.

The test article of the compound of Formula I was freshly prepared in vehicle of 100 mM acetic buffer weekly and stored under ambient conditions. The combination agent osimertinib was prepared weekly in vehicle of 0.5% HPMC and 0.1% Tween 80 weekly and stored under ambient conditions.

Female Balb/c nude mice were purchased from the Beijing Vital River Laboratory Animal Technology Co., Ltd. Mice were hosted at special pathogen-free (SPF) environment of vivarium facility and acclimated to their new environment for at least 3 days prior to initiation of any experiments. Mice were between 6-8 weeks of age at the time of implantation.

All procedures related to animal handling, care, and treatment in this study were performed according to the protocols and guidelines approved by the Institutional Animal Care and Use Committee (IACUC) of GenenDesign. Animal facility and program is operated under the standard of Guide for the Care and Use of Laboratory Animals (NRC, 2011) and accredited by the Association for Assessment and Accreditation of Laboratory Animal Care (AAALAC). Specifically, all portions of this study performed at GenenDesign adhered to the study protocols reviewed and approved by IACUC and applicable standard operating procedures (SOPs).

Preparation of PDX model

The LUN2355-128-33 model was established for pre-clinical efficacy study at GenenDesign (Shanghai, China). This PDX model was derived from a 49-year-old male Chinese NSCLC patient. The EGFR mutations in the PDX model was confirmed by whole exome sequencing and PCR sequencing. Tumor fragments harvested from the PDX model were implanted subcutaneously in the right flanks of female Balb/c nude mice. Mice were anesthetized with isoflurane and anesthesia was maintained throughout the implantation procedure. Mouse skin was cleaned with appropriate surgical scrub and alcohol over the right flank. Aseptic surgical procedures were used. A small skin incision was made using the sharp end of the trochar and a 1.5 cm subcutaneous pocket along the right lateral chest wall was formed by blunt dissection with the stylet of a 10-12 g trochar needle. Tumor fragments (15 -30 mm³) were placed into the trochar needle and advanced into the subcutaneous pocket in the right flank. Trochar incision was closed with suture or a wound clip that was removed one week after closure.

When tumor sizes reached 150-250 mm³ in volume, tumor-bearing mice were randomly divided into study groups with 8 mice in each group. The randomization date was denoted as treatment day 0.

### Treatment

Treatment started on the same day of randomization. The treatment start day was denoted as treatment day 0. Mice were dosed by oral administration of vehicle control solution, the compound of Formula I alone at 5 mg/kg BID, the compound of Formula I alone at 15 mg/kg QD, and osimertinib alone at 15 mg/kg QD. Two additional groups received combination treatments of the compound of Formula I and osimertinib, with one group dosed with the combination of the compound of Formula I at 5 mg/kg BID and osimertinib at 15 mg/kg QD, and the other group dosed with the combination of the compound of Formula I at 15 mg/kg QD and osimertinib at 15 mg/kg QD. The dosing volume was 5 mL/kg for each compound and the interval of BID regimen was 8 hours. Osimertinib was dosed one hour after the dosing of the compound of Formula 1 QD or after the first dose of the BID regimen in the combination groups.

### Results

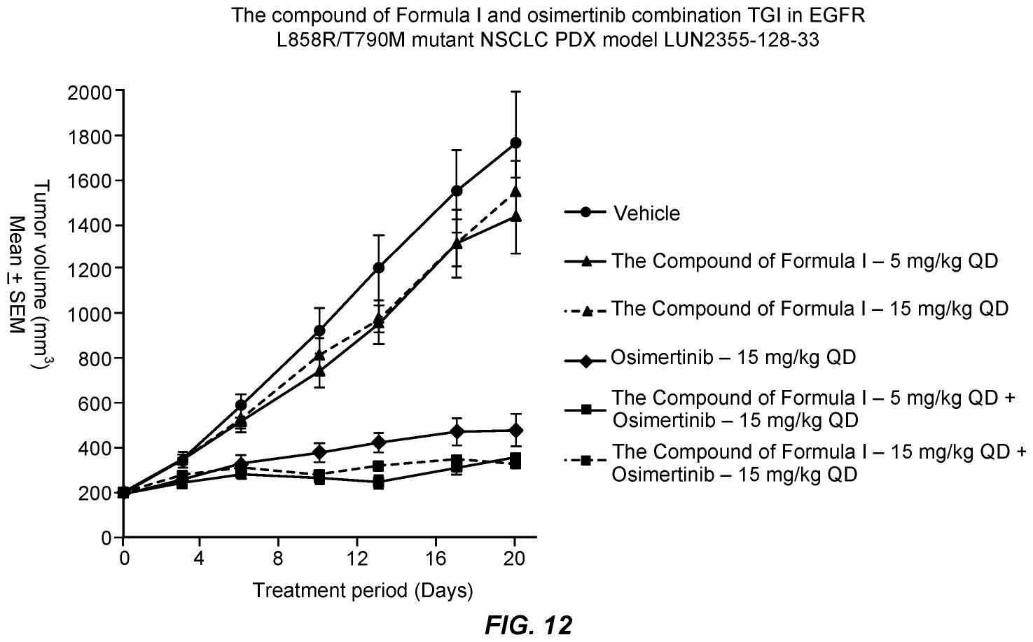

FIG. 12 shows a graph of tumor volume over a period of treatment time with a regimen of the compound of Formula I alone, osimertinib alone, and the combination of the compound of Formula I and osimertinib in EGFR L858R/T790M mutant NSCLC PDX model LUN2355 -128-33. No significant body weight change was observed in the control and treatment groups.

### Conclusion

As shown in FIG. 12, the combination of the compound of Formula I and osimertinib demonstrated superior tumor growth inhibition relative to treatment with the compound of Formula I alone or treatment with osimertinib alone in EGFRL858R/T790M mutant NSCLC PDX model LUN2355-128-33.

### Example 11—Combination Therapy of the Compound of Formula I with Osimertinib in EGFR^{ex19del} mutant CDX model HCC827/ER1

### Materials

The vehicle/control article, 100 mM acetic acid in deionized water, with pH adjustment to 4.8-5.0, was prepared and stored under ambient conditions throughout the 28-day administration in mice.

The test article of the compound of Formula I was freshly prepared in vehicle of 100 mM acetic buffer weekly and stored under ambient conditions. The combination agent osimertinib was prepared weekly in vehicle of 0.5% HPMC and 0.1% Tween 80 weekly and stored under ambient conditions.

Female Balb/c nude mice were purchased from the SPF (Beijing) Laboratory Animal Technology Co, Ltd. Mice were between 9-11 weeks of age at the time of implantation. Mice were hosted at special pathogen-free (SPF) environment of vivarium facility and acclimated to their new environment for at least 3 days prior to initiation of any experiments.

All procedures related to animal handling, care, and treatment in this study were performed according to guidelines approved by the Institutional Animal Care and Use Committee (IACUC) of Crown Bioscience (Beijing, China). During the study, the care and use of animals were conducted in accordance with the regulations of the Association for Assessment and Accreditation of Laboratory Animal Care (AAALAC). In addition, all portions of this study performed at Crown Bioscience (Beijing, China) adhered to the study protocols approved by the study director and applicable standard operating procedures (SOPs).

Preparation ofxenograft model

Human lung cancer HCC827 cells were purchased from ATCC. The EGFR exon 19 deletion mutant and erlotinib-resistant MET amplified HCC827/ER1 cell line (Crown Bioscience UK) was derived from HCC827 (ATCC) at Crown Bioscience, by culturing the cells in the presence of escalating concentrations of erlotinib. HCC827/ER1 cells were cultured in medium containing RPMI-1640 plus 10% Fetal Bovine Serum (FBS) and 42 μM erlotinib at 37° C. in an atmosphere of 5% CO₂ in air. The medium was renewed routinely, and tumor cells were sub-cultured every 3 to 5 days ata confluence of 80% by trypsin-EDTA. The cells growing in an exponential growth phase were harvested and counted for inoculation. Briefly, 100 μL cell suspensions containing 5×10⁶ HCC827/ER1 tumor cells mixed with 50% Matrigel were implanted into the right flank of the mouse subcutaneously using a syringe. Animal health and tumor growth were monitored daily after implantation. Tumor volume was measured twice a week by caliper when xenograft tumors were palpable and measurable.

When tumor sizes reached a mean of approximately 145 mm³ (range of 104-189 mm³), tumor-bearing mice were randomly divided into study groups with 8 mice in each group. The randomization date was denoted as treatment day 0.

### Treatment

Treatment started on the same day of randomization. The treatment start day was denoted as treatment day 0. Mice were dosed by oral administration of vehicle control solution, the compound of Formula I alone at 30 mg/kg QD, and osimertinib alone at 15 mg/kg QD. Based on a previously executed tolerability study that identified the optimal doses of the compound of Formula I when combined with osimertinib, the compound of Formula I dose level was reduced from 30 mg/kg QD to 15 mg/kg QD for the combination therapy. One additional group received the combination treatment of the compound of Formula I and osimertinib, with dosing of the compound of Formula I at 15 mg/kg QD and dosing of osimertinib at 15 mg/kg QD. The dosing volume was 5 mL/kg for each compound. Osimertinib was dosed one hour after the dosing of the compound of Formula I QD in the combination group. The study was terminated on treatment day 28 as being defined in the study protocol.

### Results

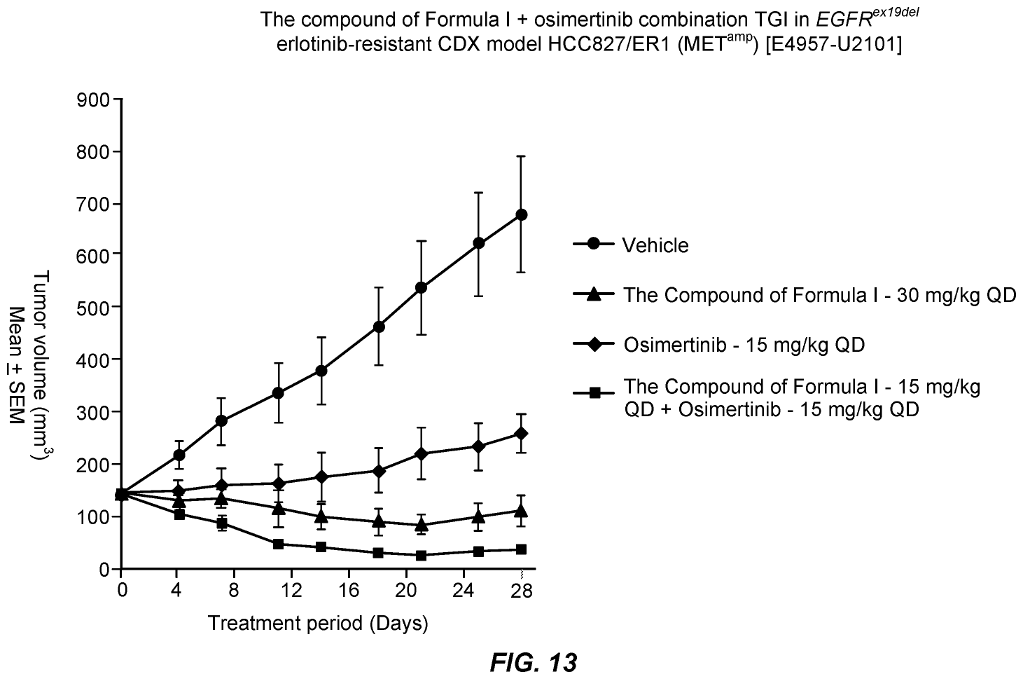

FIG. 13 shows a graph of tumor volume over a period of treatment time with a regimen of the compound of Formula I alone, osimertinib alone, and the combination of the compound of Formula I and osimertinib in EGFR^{ex19del} mutant CDX model HCC827/ER1. No significant body weight change was observed in the control and treatment groups.

### Conclusion

As shown in FIG. 13, the combination of the compound of Formula I and osimertinib demonstrated superior tumor growth inhibition relative to treatment with the compound of Formula I alone or treatment with osimertinib alone in EGFR^{ex19del} mutant CDX model HCC827/ER1.

### Example 12—Combination Therapy of the Compound of Formula I with Cetuximab in RAS/RAF wild type PDX model CRC049

### Materials

The vehicle/control article, 100 mM acetic acid in deionized water, with pH adjustment to 4.8-5.0, was prepared and stored under ambient conditions throughout the 28-day administration in mice.

The test article of the compound of Formula I was freshly prepared in vehicle of 100 mM acetic buffer weekly and stored under ambient conditions. The combination agent cetuximab was prepared in PBS and stored at 2-8° C.

Female Balb/c nude mice were purchased from the Beijing Vital River Laboratory Animal Technology Co., Ltd. Mice were hosted at special pathogen-free (SPF) environment of vivarium facility and acclimated to their new environment for at least 3 days prior to initiation of any experiments. Mice were between 6-8 weeks of age at the time of implantation.

All procedures related to animal handling, care, and treatment in this study were performed according to the protocols and guidelines approved by the Institutional Animal Care and Use Committee (IACUC) of GenenDesign. Animal facility and program is operated under the standard of Guide for the Care and Use of Laboratory Animals (NRC, 2011) and accredited by the Association for Assessment and Accreditation of Laboratory Animal Care (AAALAC). Specifically, all portions of this study performed at GenenDesign adhered to the study protocols reviewed and approved by IACUC and applicable standard operating procedures (SOPs).

Preparation of PDX model

CRC049 PDX model was established for efficacy study at GenenDesign. This PDX model was derived from a 79-years old male Chinese CRC patient. KRAS, NRAS, and HRAS, ARAF, BRAF, and RAF1 genes in the PDX model were analyzed by whole exome sequencing and PCR sequencing. Tumor fragments harvested from the PDX mouse model were implanted subcutaneously in the right flanks of female Balb/c nude mice. Mice were anesthetized with isoflurane and anesthesia was maintained throughout the implantation procedure. Mouse skin was cleaned with appropriate surgical scrub and alcohol over the right flank. Aseptic surgical procedures were used for implantation. A small skin incision was made using the sharp end of the trochar and a 1.5 cm subcutaneous pocket along the right lateral chest wall formed by blunt dissection with the stylet of a 10-12 g trochar needle. Tumor fragments (15-30 mm³) were placed into the trochar needle and advanced into the subcutaneous pocket in the right flank. Trochar incision was closed with suture or a wound clip that were removed one week after closure. When tumor sizes reached 143-249 mm³ in volume, mice were randomly divided into study groups with 8 mice in each group. The randomization date was denoted as treatment day 0.

### Treatment

Treatment started on the day of randomization. The treatment start day was denoted as treatment day 0. Mice were dosed by oral administration of vehicle control solution, cetuximab alone at 20 mg/kg BIW, and the compound of Formula I alone at 30 mg/kg QD. One additional group received the combination treatment of the compound of Formula I and cetuximab, with dosing of the compound of Formula I at 30 mg/kg QD, and dosing of cetuximab at 20 mg/kg BIW. The dosing volume was 5 mL/kg for each compound. Cetuximab was dosed at one hour after the dose of the compound of Formula I at 30 mg/kg QD in the combination group. The study was terminated on day 28 as defined in the study protocol.

### Results

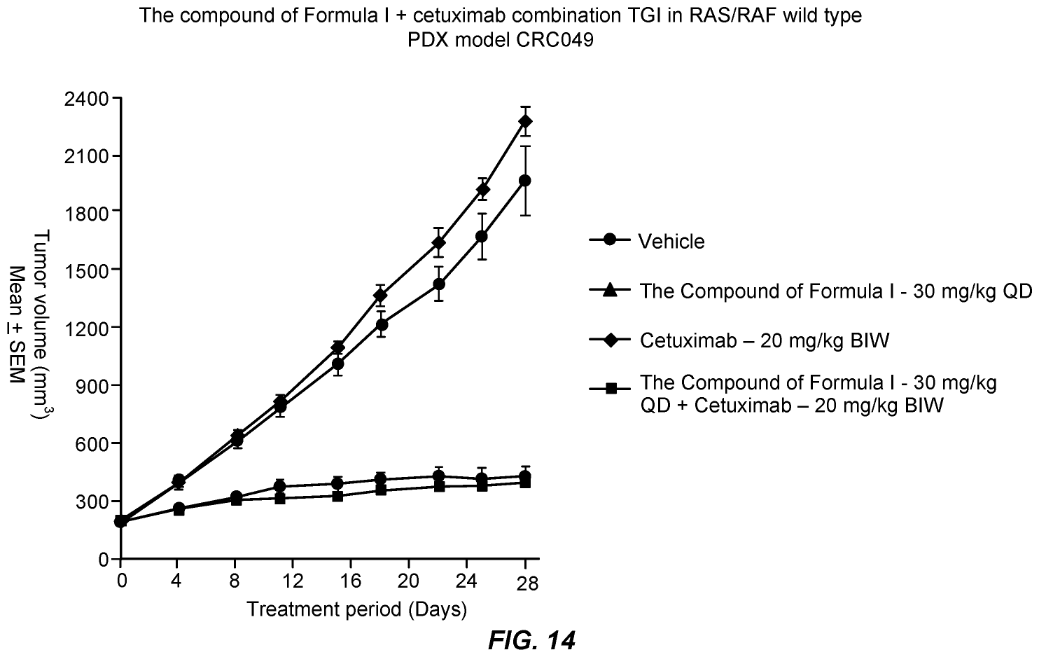

FIG. 14 shows a graph of tumor volume over a period of treatment time with a regimen of the compound of Formula I alone, cetuximab alone, and the combination of the compound of Formula I and cetuximab in RAS/RAF wild type PDX model CRC049. No significant body weight change was observed in the control and treatment groups.

### Conclusion

As shown in FIG. 14, the combination of the compound of Formula I and cetuximab demonstrated superior tumor growth inhibition relative to treatment with the compound of Formula I alone or treatment with cetuximab alone in RAS/RAF wild type PDX model CRC049.

### Example 13—Combination Therapy of the Compound of Formula I with Cetuximab in RAS/RAF Wild Type HPV-Negative CDX Model FaDu

### Materials

The vehicle/control article, 100 mM acetic acid in deionized water, with pH adjustment to 4.8-5.0, was prepared and stored under ambient conditions throughout the 28-day administration in mice.

The test article Formula 1 was freshly prepared in vehicle of 100 mM acetic buffer weekly and stored under ambient conditions. The combination agent cetuximab (5 mg/mL, stored at 2-8° C.) was diluted with saline to 3 mg/mL before each dosing.

Female Balb/c nude mice were purchased from the Beijing Vital River Laboratory Animal Technology Co., Ltd. Mice were between 6-8 weeks of age at the time of implantation. Mice were hosted at special pathogen-free (SPF) environment of vivarium facility and acclimated to their new environment for at least 3 days prior to initiation of any experiments.

All procedures related to animal handling, care, and treatment in this study were performed according to guidelines approved by the Institutional Animal Care and Use Committee (IACUC) of WuXi AppTec. During the study, the care and use of animals were conducted in accordance with the regulations of the Association for Assessment and Accreditation of Laboratory Animal Care (AAALAC). In addition, all portions of this study were performed at WuXi AppTec and adhered to the study protocols approved by the study director and applicable standard operating procedures (SOPs).

### Preparation of Xenograft Model

FaDu was a RAS/RAF wildtype human head and neck squamous cell cancer (pharyngeal cancer) cell line. The FaDu cell line was purchased from the American Type Culture Collection (ATCC® HTB-43™). FaDu cells were cultured in medium containing EMEM plus 10% Fetal Bovine Serum (FBS) and 1% Antibiotic-Antimycotic (AA) at 37° C. in an atmosphere of 5% CO₂ in air. The medium was renewed every 2 to 3 days and tumor cells were routinely sub-cultured at a confluence of 80-90% by trypsin-EDTA. The cells growing in an exponential growth phase were harvested and counted for inoculation.

FaDu tumor cells were implanted into mice subcutaneously. 200 μL cell suspensions containing 5×10⁶ tumor cells were subcutaneously implanted into the right flank of mouse using a syringe. Animal health and tumor growth were monitored daily. Tumor volume was measured twice a week by caliper when tumors were palpable and measurable. When tumor volumes reached a mean of 153 mm³ (range of 75-217 mm³), tumor-bearing mice were randomized into different groups with 8 mice in each group. The randomization date was denoted as treatment day 0.

### Treatment

Treatment started on the day after randomization. The treatment start day was denoted as treatment day 1. Mice were dosed by oral administration of vehicle control solution, cetuximab alone at 30 mg/kg Q3D and the compound of Formula I alone at 10 mg/kg BID. One additional group received the combination treatment of the compound of Formula I and cetuximab, with dosing of the compound of Formula I at 10 mg/kg BID and dosing of cetuximab at 30 mg/kg Q3D. The dosing volume was 5 mL/kg for the compound of Formula I and 10 mL/kg for Cetuximab. Interval of BID regimen for the compound of Formula I was 8 hours. Cetuximab was dosed one hour after the first dose of the compound of Formula I BID dose in the combination group. The study was terminated on day 28 as being defined in the study protocol.

### Results

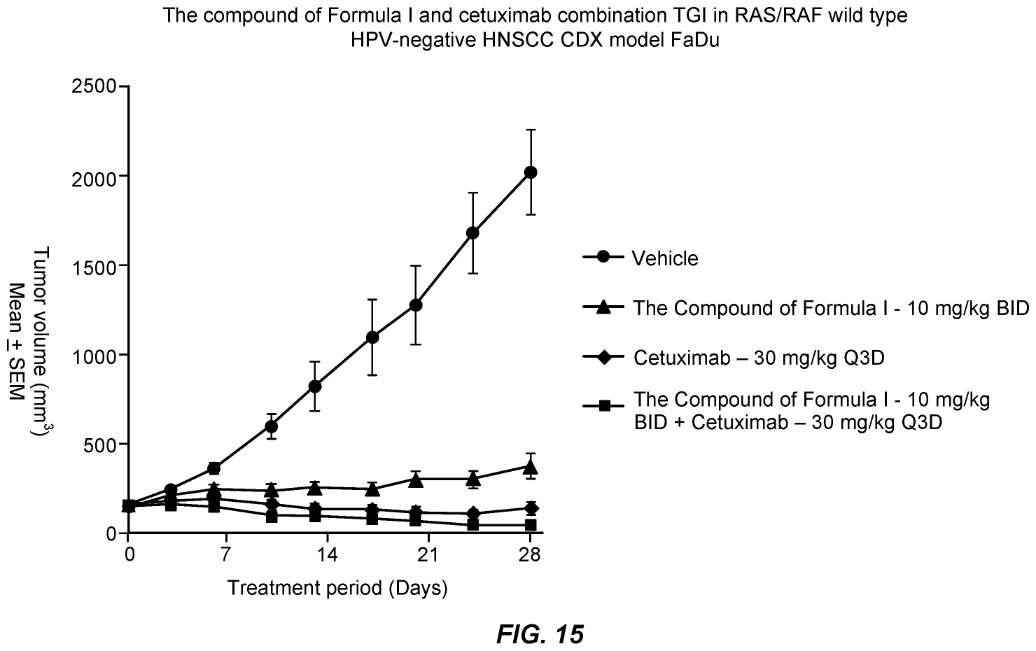

FIG. 15 shows a graph of tumor volume over a period of treatment time with a regimen of the compound of Formula I alone, cetuximab alone, and the combination of the compound of Formula I and cetuximab in RAS/RAF wild type HPV-negative HNSCC CDX model FaDu. No significant body weight change was observed in the control and treatment groups.

### Conclusion

As shown in FIG. 15, the combination of the compound of Formula I and cetuximab demonstrated superior tumor growth inhibition relative to treatment with the compound of Formula I alone or treatment with cetuximab alone in RAS/RAF wild type HPV-negative HNSCC CDX model FaDu.

Although the foregoing embodiments have been described in some detail by way of illustration and Examples for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method of treating a subject having cancer comprising administering to the subject a therapeutically effective amount of a compound of Formula I or its pharmaceutically acceptable salt: 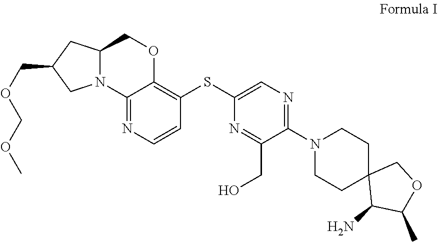 [SMILES: COCOC[C@H]1C[C@H]2COc3c(Sc4cnc(N5CCC6(CC5)CO[C@@H](C)[C@H]6N)c(CO)n4)ccnc3N2C1]
in combination with an EGFR inhibitor.

2. The method of claim 1, wherein the EGFR in the subject is expressed constitutively.

3. The method of claim 1, wherein the cancer comprises an EGFR mutation selected from EGFR gene copy gain, EGFR gene amplification, chromosome 7 polysomy, EGFR L858R, EGFR exon 19 deletions/insertions (e.g., E746_A750del, E746_T751delinsI, E746_T751delinsIP, E746_S752delinsA, E746_S752delinsV, E746_S752delinsV, L747_S752del, L747_T751del, and L747_P753 delinsS), EGFR L861Q, EGFR G719C, EGFR G719S, EGFR G719A, EGFR V765A, EGFR T783A, EGFR exon 20 insertions (e.g., N771dup, N771_H773 dup, and P772_H773 dup), EGFR splice variants (e.g., Viii, Vvi, and Vii), EGFR A289D, EGFR A289T, EGFR A289V, EGFR G598A, EGFR G598V, EGFR T790M, and EGFR C797S.

4. The method of claim 1 or 2, wherein the cancer lung cancer.

5. The method of claim 1 or 2, wherein the cancer is an adenocarcinoma.

6. The method of claim 1 or 2, wherein the cancer is pancreatic ductal adenocarcinoma (PDAC).

7. The method of any one of claims 1 to 6, wherein the EGFR inhibitor is selected from osimertinib, dacomitinib, lazertinib, nazartinib, neratinib, mobocertinib, afatinib, erlotinib, gefitinib, lapatinib, lifirafenib, amivantamab, cetuximab, panitumumab, necitumumab, mirzotamab clezutodax, nimotuzumab and vandetanib.

8. The method of any one of claims 1 to 6, wherein the EGFR inhibitor is osimertinib.

9. The method of any one of claims 1 to 6, wherein the EGFR inhibitor is erlotinib.

10. The method of any one of claims 1 to 6, wherein the EGFR inhibitor is gefitinib.

11. The method of any one of claims 1 to 6, wherein the EGFR inhibitor is lapatinib.

12. The method of any one of claims 1 to 6, wherein the EGFR inhibitor is neratinib.

13. The method of any one of claims 1 to 6, wherein the EGFR inhibitor is afatnib.

14. The method of any one of claims 1 to 13, wherein the method comprises administering a third MAPK pathway inhibitor.

15. The method of any one of claims 1 to 14, wherein the administration is oral.

16. The method of any one of claims 1 to 15, wherein the dosing of the compound of Formula I is in a range from 20 mg to 400 mg daily.

17. The method of any one of claims 1 to 16, wherein the dosing of the EGFR inhibitor is in a range from 1 mg to 1500 mg daily.

18. A method of treating lung cancer in a subject comprising orally administering to the subject a therapeutically effective amount of a compound of Formula I or its pharmaceutically acceptable salt: 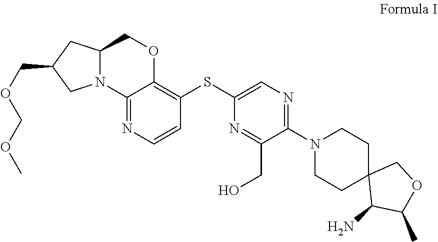 [SMILES: COCOC[C@H]1C[C@H]2COc3c(Sc4cnc(N5CCC6(CC5)CO[C@@H](C)[C@H]6N)c(CO)n4)ccnc3N2C1]
in combination with osimertinib.

19. The method of claim 18, wherein the compound of Formula I is administered once or twice daily.

20. The method of claim 18 or 19, wherein osimertinib is administered once or twice daily.

21. The method of any one of claims 18 to 20, wherein the subject is a human.

22. The method of any one of claims 18 to 21, wherein the lung cancer is non-small cell lung carcinoma.

23. The method of any one of claims 18 to 22, wherein the lung cancer has an EGFR mutation.

24. A kit comprising a compound of Formula I or a pharmaceutically acceptable salt thereof and an EGFR inhibitor.

25. The kit of claim 24, wherein the compound of Formula I and the EGFR inhibitor are in separate packages.

26. The kit of claim 24 or 25, wherein the kit further comprises instructions to administer the contents of the kit to a subject for the treatment of cancer.

27. The kit of any of claims 24 to 26, wherein the EGFR inhibitor is one or more of osimertinib, dacomitinib, lazertinib, nazartinib, neratinib, mobocertinib, afatinib, erlotinib, gefitinib, lapatinib, lifirafenib, amivantamab, cetuximab, panitumumab, necitumumab, mirzotamab clezutoclax, nimotuzumab and vandetanib.

28. A method of treating cancer in a subject comprising orally administering to the subject a therapeutically effective amount of a compound of Formula I or a pharmaceutically acceptable salt thereof: 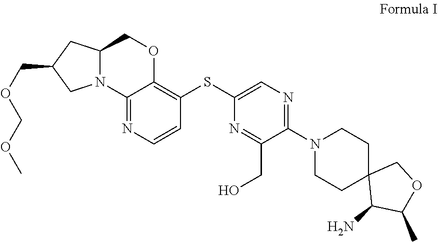 [SMILES: COCOC[C@H]1C[C@H]2COc3c(Sc4cnc(N5CCC6(CC5)CO[C@@H](C)[C@H]6N)c(CO)n4)ccnc3N2C1]
in combination with cetuximab.

29. The method of claim 28, wherein cetuximab is administered weekly.

30. The method of claim 28 or 29, wherein cetuximab is administered every other week.

31. The method of any one of claims 28 to 30, wherein the cancer is squamous cell head and neck cancer (SCCHN).

32. The method of any one of claims 28 to 30, wherein the cancer is colorectal cancer or head and neck squamous cell carcinoma.

33. The method of any one of claims 28 to 30, wherein the cancer is HPV negative.

34. The method of any one of claims 28 to 30, wherein the cancer is wtKRAS/wtNRAS/wtBRAF.

35. The method of any one of claims 28 to 30, wherein the cancer does not have a mutation in KRAS, NRAs, or BRAF.

36. The method of any one of claims 28 to 30, wherein the cancer is pancreatic ductal adenocarcinoma (PDAC).

37. The method of any one of claims 28 to 36, wherein the subject is a human.

38. The method of any one of claims 1 to 37, wherein the compound of Formula I, or pharmaceutically acceptable salt thereof, is administered at a dose between about 20 mg and about 260 mg per day.

39. The method of any one of claims 1 to 38, wherein the compound of Formula I, or pharmaceutically acceptable salt thereof, is administered at a dose between about 20 mg and about 60 mg per day.

40. The method of any one of claims 1 to 39, wherein the compound of Formula I, or pharmaceutically acceptable salt thereof, is administered at a dose of about 40 mg per day or about 60 mg per day.

41. The method of any one of claims 1 to 37, wherein the compound of Formula I, or pharmaceutically acceptable salt thereof, is administered at a dose between about 10 mg and about 100 mg twice per day.

42. The method of any one of claims 1 to 37, wherein the compound of Formula I, or pharmaceutically acceptable salt thereof is administered at a dose between about 20 mg and about 80 mg twice per day.

43. The method of any one of claims 1 to 42, wherein the compound of Formula I, or a pharmaceutically acceptable salt thereof, is formulated as a pharmaceutical composition.

44. The method of claim 43, wherein the compound, or a pharmaceutically acceptable salt thereof, is formulated as an oral composition.

45. The method of any one of claims 1 to 44, wherein the compound of Formula I, or a pharmaceutically acceptable salt thereof, is administered once or twice a day.

46. The method of any one of claims 1 to 45, wherein the compound of Formula I, or a pharmaceutically acceptable salt thereof, is administered over a continuous 28-day cycle.

47. The method of any one of claims 1 to 46, wherein the compound of Formula I, or a pharmaceutically acceptable salt thereof, is administered once a day in the amount of about 10 mg to about 140 mg.

48. The method of any one of claims 1 to 47, wherein the compound of Formula I, or a pharmaceutically acceptable salt thereof, is administered once a day for a 3 -week cycle, comprising 2 weeks of administration of the compound followed by 1 week of no administration of the compound.

49. The method of any one of claims 1 to 47, wherein the compound of Formula I, or a pharmaceutically acceptable salt thereof, is administered once a day for a 4-week cycle, comprising 3 weeks of administration of the compound followed by 1 week of no administration of the compound.

50. The method of any one of claims 1 to 49, wherein the compound of Formula I, or a pharmaceutically acceptable salt thereof, is administered over a period of 6 weeks.

51. The method of any one of claims 1 to 49, wherein the compound of Formula I, or a pharmaceutically acceptable salt thereof, is administered over a period of 8 weeks.

52. The method of any one of claims 1 to 51, wherein the compound of Formula I, or a pharmaceutically acceptable salt thereof, is administered 3 times a week.

53. The method of claim 52, wherein the compound of Formula I, or a pharmaceutically acceptable salt thereof, is administered on day 1, day 3, and day 5 of the week.

54. The method of any one of claims 1 to 53, wherein the compound of Formula I, or a pharmaceutically acceptable salt thereof, is administered 4 times a week.

55. The method of claim 54, wherein the compound of Formula I, or a pharmaceutically acceptable salt thereof, is administered for a 3-week cycle, comprising 2 weeks of administration of the compound followed by 1 week of no administration of the compound.

56. The method of claim 54, wherein the compound of Formula I, or a pharmaceutically acceptable salt thereof, is administered for a 4-week cycle, comprising 3 weeks of administration of the compound followed by 1 week of no administration of the compound.

57. The method of any one of claims 1 to 56, wherein the compound of Formula I, or a pharmaceutically acceptable salt thereof, is administered twice a day, two days per week.

58. The method of any one of claims 1 to 57, wherein the compound of Formula I, or a pharmaceutically acceptable salt thereof, is administered over a period of 8 weeks.

59. The method of claim 57 or 58, wherein the compound of Formula I, or a pharmaceutically acceptable salt thereof, is administered on day 1 and day 2 of each week.

60. The method of any one of claims 1 to 59, wherein the cancer is selected from lung cancer, stomach cancer, liver cancer, colon cancer, kidney cancer, breast cancer, pancreatic cancer, pancreatic ductal adenocarcinoma (PDAC), juvenile myelomonocytic leukemia, neurolastoma, melanoma, and acute myeloid leukemia.
